(19) United States
(12) Patent Application Publication
(10) Pub. No.: US 2005/0244382 A9
(43) Pub. Date: Nov. 3, 2005

(54) Compositions including ammonia oxidizing bacteria and methods of using same

(76) Inventor: David R. Whitlock, Watertown, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 10/501,415

(22) PCT Filed: Jan. 13, 2003
(86) PCT No.: PCT/US03/00995

§ 371 Date: Dec. 29, 2004

Related U.S. Application Data
(60) Provisional application No. 60/347,760, filed on Jan. 11, 2002.
(60) Provisional application No. 60/375,680, filed on Apr. 26, 2002.
(60) Provisional application No. 60/413,667, filed on Sep. 24, 2002.

(51) Int. Cl. A61K 45/00; C12N 1/21

(52) U.S. Cl. 424/93.4; 435/252.1; 435/252.3

(57) ABSTRACT

A preparation and method for treating a subject how had developed or is at risk of developing at least one of high blood pressure, Alzheimer's Disease, obesity, and Diabetes Type II, Sickle Cell Anemia, Preeclampia, Sudden Infant Death Syndrome, or Vascular disease comprising positioning ammonia oxidizing bacteria close proximity of a surface of the subject, of nitric oxide and nitric oxide precursors using ammonia oxidizing bacteria.

### FIELD OF INVENTION

The present invention relates to a composition including ammonia oxidizing bacteria to increase production of nitric oxide and nitric oxide precursors on the surface of a subject and methods of using same to reduce blood pressure, treat Alzheimer's Disease, treat obesity, and treat diabetes Type 2 in a subject, specifically by administering nitric oxide to the subject.

### BACKGROUND

Beneficial bacteria have been utilized to suppress the growth of pathogenic bacteria. Bacteria and other microorganisms are ubiquitous in the environment. The discovery of pathogenic bacteria and the germ theory of disease has had a tremendous effect on health and disease states. Bacteria are a normal part of the intestinal contents of all living things. These bacteria are not pathogenic under normal conditions, and in fact improve health by rendering the normal intestinal contents less hospitable for disease causing organisms. This is accomplished in a number of ways: nutrients are consumed, leaving less for pathogens; conditions are produced, such as pH, oxygen tension, which are not hospitable for pathogens; compounds are produced that are toxic to pathogens; pathogens are consumed as food by these microorganisms; less physical space remains available for pathogens; and specific binding sites are occupied leaving fewer for pathogens. The presence of these desirable bacteria is seen as useful in preventing disease states.

Fermentation of food products has been done to substitute a desired non-pathogenic strain for potential spoilage or pathogenic organisms. Brewed beverages, wine, pickled food, fermented milk products including cheese, yogurt, buttermilk, sausage are all examples where desired microorganisms are deliberately inoculated into food products under conditions that favor their growth and inhibit the growth of spoilage and pathogenic strains. U.S. Patents disclosing the use of specific bacteria to inhibit the growth of harmful bacteria include: U.S. Pat. No. 3,984,575 issued to Farr Oct. 5, 1976; U.S. Pat. No. 4,689,226 issued to Nurmi, et al. Aug. 25, 1987; U.S. Pat. No. 5,322,686 issued to Grahn, et al. Jun. 21, 1994; U.S. Pat. No. 5,451,400 issued to Stern, et al. Sep. 19, 1995; U.S. Pat. No. 5,604,127 issued to Nisbet, et al. Feb. 18, 1997; and U.S. Pat. No. 5,807,546 issued to Stern, et al. Sep. 15, 1998.

U.S. Pat. No. 5,176,911 issued to Tosi, et al. Jan. 5, 1993 discloses the use of specific bacteria recovered from healthy asymptomatic patients and characterized in the laboratory as a preventative and curative topical application to the vaginal area of women suffering from vaginal yeast infections.

It is known that nitric oxide gas may be administered, and is also generated in nasal passages during inhalation and drawn into the lung along with inhaled air. Thus nitric oxide is absorbed in the lung where it attaches to hemoglobin and forms S-nitrosolated hemoglobin. This is a major source of S-nitrosolated hemoglobin producing systemic effects in the body. The following United States patents disclose various physiological effects of nitric oxide inhalation: U.S. Pat. No. 5,427,797 issued to Frostell, et al. Jun. 27, 1995; U.S. Pat. No. 5,765,548 issued to Perry Jun. 16, 1998; and U.S. Pat. No. 5,904,938 issued to Zapol, et al. May 18, 1999.

U.S. Pat. No. 5,519,020 issued to Smith, et al. May 21, 1996, discloses the use of nitric oxide releasing materials, placed in close proximity to wounds to enhance healing through a variety of mechanisms. A polymeric material is used to control the rate at which nitric oxide is released because nitric oxide may be toxic and injurious in excessive doses.

U.S. Pat. No. 5,646,181 issued to Fung, et al. Jul. 8, 1997 discloses topical medications containing organic nitric oxide releasing compounds that when topically applied release nitric oxide in sufficient quantities to treat impotence without producing systemic side effects such as hypotension.

U.S. Pat. No. 5,648,101 issued to Tawashi Jul. 15, 1997 discloses products that liberate nitric oxide through reaction of an inorganic nitrite and a ferrous metal salt. These products may be ingested, applied topically, taken as suppositories, applied as transdermal patches, and used in osmotic pumps.

U.S. Pat. No. 5,891,472 issued to Russell Apr. 6, 1999 discloses the use of topically applied nitric oxide donors for the treatment of equine laminitis.

U.S. Pat. No. 5,895,658 issued to Fossel Apr. 20, 1999, discloses the use of topically applied L-arginine, a substrate for production of nitric oxide form nitric oxide synthase to cause local vasodilatation of the skin for the purpose of producing beneficial effects such as warming of cold or cool tissues, growth of hair on the scalp, healing of leg ulcers secondary to diabetes or confinement to bed, as well as beneficial effects through restoration of natural mechanisms based on improvement of local blood supply.

U.S. Pat. No. 5,721,278 issued to Garfield, et al. Feb. 24, 1998, discloses the use of inhibitors of nitric oxide synthesis injected into the body of a subject to inhibit ovulation, and the use of nitric oxide precursors to bring about ovulation.

U.S. Pat. 5,800,385 issued to Demopulos, et al. Sep. 1, 1998, discloses solutions including nitric oxide donors for irrigating the sites of operative wounds. The nitric oxide donors may be included in the solutions for their anti-spasm activity.

U.S. Pat. 5,858,017 issued to Demopulos, et al. Jan. 12, 1999, discloses the use of solutions containing among other things, nitric oxide donors in urological irrigation solutions.

U.S. Pat. 5,861,168 issued to Cooke, et al. Jan. 19, 1999 discloses the intramural application of nitric oxide precursors during coronary balloon angioplasty to reduce thickening of the treated vessels and to improve tolerance to the angioplasty procedure.

U.S. Pat. No. 5,278,192 issued to Fung, et al. Jan. 11, 1994, discloses using organic nitrates for continuous treatment of conditions including, angina, particularly chronic, stable angina pectoris, ischemic diseases, congestive heart failure, for controlling hypertension and/or impotence in male patients. These organic nitrates may be administered in a variety of ways including sublingual, oral and buccal tablets as well as capsules, topical creams and ointments, patches, tapes, spray and intravenous solutions.

U.S. Pat. No. 5,385,940 issued to Moskowitz Jan. 31, 1995 discloses the administering nitric oxide donors or L-arginine to act as the substrate of nitric oxide synthase during a stroke to increase nitric oxide production and so cause vasodilatation to reduce the infarct size.

U.S. Pat. No. 5,650,447 Keefer, et al. Jul. 22, 1997 discloses the use of polymers containing bound nitric oxide releasing compounds to treat restenosis when incorporated into devices such as sutures, vascular implants, stents, heart valves, drug pumps, drug-delivery catheters, self-adhering means such as endoluminal implants, liposomes, microparticles, microspheres, beads, disks or other devices.

U.S. Pat. No. 5,789,447 issued to Wink, Jr., et al. Aug. 4, 1998, discloses a method of reducing free radical induced tissue damage associated with ischemia reperfusion injury wherein the ischemia reperfusion injury is associated with a condition or disease selected from the group consisting of transplantation, trauma, inflammation, stroke, seizure, rheumatoid arthritis, atherosclerosis, cancer, dementia, diabetes, hypertensive crisis, ulcers, lupus, sickle cell anemia, ischemic bowel syndrome, pulmonary emboli, Ball's syndrome, pancreatitis, heart attack, and aging.

U.S. Pat. No. 5,814,666 issued to Green, et al. Sep. 29, 1998, disclose the use of nitric oxide releasing compounds as antimicrobial agents.

U.S. Pat. No. 6,057,367 issued to Stamler, et al. May 2, 2000 disclose using a variety of methods to manipulate nitrosative stress. These methods include using acidified nitrite as a mouth rinse and a mixture of acidified nitrite plus a thiol as a topical application. S-nitrosothiol may be applied topically or formed in situ from an inorganic nitrite, a pharmacologially acceptable acid and a thio. Pathenogenic microbes may also convert substrates to nitrosating agents which inhibit the growth of the pathenogenic microbe.

In the book, “Nitric Oxide and Infection”, Ferric C. Fang ed., Kluwer Academic/Plenum Publishers, 1999, in a chapter titled “Nitric Oxide and Epithelial Host Defense” by Nigel Benjamin and Roelf Dykhuizen, the authors disclose the relevance of nitric oxide production on the skin in normal infection control, and a salve containing acidified nitrite is effective in the treatment of tinea pedis (athlete's foot). They attribute the normal production of nitrite on the skin to the reduction of sweat nitrate to nitrite by skin bacteria.

However, many heterotrophic bacteria will reduce nitrate to nitrite, for example, E. coli. These bacteria are facultative anaerobes that normally utilize oxygen as the electron sink for their cellular respiration, but can also utilize nitrate in the absence of oxygen. All these bacteria utilize organic substrates for energy and growth and many of these bacteria can be pathogenic. In the mouth, salivary nitrate is reduced by these facultative anaerobes. These nitrate reducing bacteria are kept anaerobic by the layers of biofilm that accumulates on the tongue. In that the surface of the skin is expected to be aerobic, reduction of nitrate to nitrite should be minor. While some nitric oxide may be produced by bacterial reduction of sweat, the urea content of sweat is much higher than that of nitrate.

### SUMMARY

A need remains for a more significant source of nitric oxide that is more easily and safely stimulated.

The present invention relates to a method of treating a subject who has developed or is at risk of developing at least one of high blood pressure, Alzheimer's Disease, obesity, diabetes type II, sickle cell anemia, preeclampia, sudden infant death syndrome, or vascular disease comprising positioning ammonia oxidizing bacteria in close proximity to the subject. In one embodiment, the bacteria is selected from the group consisting of any of Nitrosomonas, Nitrosococcus, Nitrosospira, Nitrosocystis, Nitrosolobus, Nitrosovibrio, and combinations thereof.

The present invention also relates to a preparation for treating a subject who has developed or is at risk of developing at least one of high blood pressure, Alzheimer's Disease, obesity, diabetes type II, sickle cell anemia, preeclampia, sudden infant death syndrome, or vascular disease comprising an active culture of nitric oxide producing bacteria.

Another aspect of the invention is directed to a method of increasing basal nitric oxide in a subject comprising positioning ammonia oxidizing bacteria in close proximity to the subject.

Another aspect of the invention is directed to a method of treating a wound in a subject comprising applying ammonia oxidizing to a wound of the subject in an effective amount to cause the bacteria to metabolize any of ammonia, ammonium salts, or urea on the surface into any of nitric oxide, nitric oxide precursors, or combinations thereof.

### BRIEF DESCRIPTION OF THE DRAWINGS

FIG. 1 shows the prevalence of Alzheimer's Disease vs. average minimum temperature during hottest month.

FIG. 2 shows the incidence of obesity in the population of the United States verses altitude.

FIG. 3 shows the incidence of diabetes in the population of the United States verses altitude.

FIG. 4 shows the number of patents issued in the United States directed to shampoo verses year issued.

FIG. 5 shows blood pressure before during and after applying a culture to the scalp of a subject.

### DETAILED DESCRIPTION

The present invention relates to a composition including ammonia oxidizing bacteria to increase production of nitric oxide and/or nitric oxide precursors in close proximity to a surface of a subject and methods for treating diseases such as Heart Disease, Alzheimer's Disease, Obesity and Diabetes Type 2 in a subject by administering nitric oxide (NO) to the subject. “Subject,” as used herein, shall mean a human or vertebrate animal including, but not limited to, a dog, cat, horse, cow, pig, sheep, goat, chicken, primate, e.g., monkey, rat, and mouse. According to an embodiment of the invention, nitric oxide, a nitric oxide precursor, and or a nitric oxide releasing compound may be positioned in close proximity to a surface of a subject to treat heart disease, treat Alzheimer's Disease, treat obesity, and treat diabetes 2. The term “treat” is used herein to mean prevent or retard the onset of a disease or disorder as well as to retard or stop the progression of disease or disorder after its onset. Nitric oxide may be topically applied, inhaled, and/or injected into the body.

More specifically, in one embodiment, applying a composition of an ammonia oxidizing bacteria to skin during or after bathing to metabolize urea and other components of perspiration into nitrite and ultimately into Nitric Oxide (NO) results in a natural source of NO. One aspect of the present invention causes topical nitric oxide release at or near the surface of the skin where it can diffuse into the skin and have local as well as systemic effects. This naturally produced nitric oxide can then participate in the normal metabolic pathways by which nitric oxide is utilized by the body. Adding urea or ammonium salts to the skin provides additional substrates that these bacteria utilize to form nitrite. As used herein, the phrase “near the surface” is defined as adjacent to or in close proximity to, but need not be in contact with the surface.

Surprisingly, it has been discovered in one embodiment of the invention that a significant source of nitric oxide are the autotrophic ammonia oxidizing bacteria living on the scalp and utilizing the urea in sweat as the substrate for nitric oxide production. Bathing with soap and running hot water is a modern custom. Prior to the development of soap and interior plumbing with running hot water, bathing was difficult and unpleasant. In places without natural bodies of water, even that option was unavailable to early humans. Without bathing, people would build up a layer of biofilm, comprised of bacteria living on the skin and subsisting on sweat residues. I have found through my own experiments, that when such a biofilm contains autotrophic ammonia oxidizing bacteria, objectionable body odor does not develop, even after several months of non-bathing in summer.

Nitric Oxide is a small molecule that diffuses rapidly through the skin into the capillaries of the skin. Vasodilatation of these capillaries would occur, as well as diffusion of NO into the blood where it may be transported to other regions of the body. Dilatation of the capillaries at the skin surface enhances blood flow to, and hence heat loss from, the skin during periods of exercise.

Heart disease and other vascular diseases are a significant cause of death in the developed world. Vascular diseases also cause significant reductions in quality of life for those afflicted. Significant medical resources are devoted to prevention, treatment and research into the causes of these forms of disease.

Exercise has long been touted as having protective effects on the heart, the vascular system, and on health in general. Numerous studies and reports have shown an inverse correlation between exercise and death from heart disease. Curiously the protective effects of exercise on the vascular system are sometimes seen to be lower at more vigorous activity levels. This diminished protective effect of more vigorous physical activity is not observed in all studies but has been observed for both heart disease and stroke. A recent study, “Physical Activity and Stroke Incidence The Harvard Alumni Health Study,” by I-Min Lee, et al. (Stroke. 1998;29:2049-2054) showed a U shaped curve of stroke incidence verses intensity of exercise. Walking was also observed to reduce stroke incidence independent of other forms of exercise. The authors were unable to explain these observations, nor has a satisfactory explanation of these observations yet been made.

Death rates due to heart disease often show significant seasonal variation. A recent article “Seasonal Variation in Chronic Heart Failure Hospitalizations and Mortality in France”, Fabrice Boulay, MD, et al. (Circulation. 1999;100:280-286.) shows pronounced increases in mortality during the winter months and declines during the summer months over a 6 year period. This study which covered the entire French population, showed a peak monthly average for January that was 20% above the yearly average. The monthly minimum was 15% below the average in August. This pattern is visible each and every year included in the study but no satisfactory explanation for this data is provided.

Diet, smoking, exercise, control of high blood pressure, being married, personality type, genetic factors, viral infections, moderate alcohol consumption have all been shown to affect rates of heart and vascular diseases. With so many factors being important it is very difficult to find the proper controls to correct for known as well as potential unknown confounding factors. I have found that another factor, which is easily controlled, may explain some of the discrepancy between different rates of vascular disorders.

Physical activity induces a number of physiological changes. As physical exertion increases, heart and respiration rate increase to supply fuel and oxygen to the cells producing work. Since this production of work is not 100% efficient, metabolic heat also increases and must be dissipated. The body increases sweat production to dissipate this heat through evaporative cooling.

While Western medicine has focused on the prompt physiological effects of exercise, sweating per se also has proponents. Raising the ambient temperature, as in a sauna, has been claimed to have salutary effects on one's health. In fact the use of high temperatures to induce sweating has been a common component of personal hygiene in many cultures prior to the introduction of soap and running (hot) water. The Turkish hammam, the Finnish sauna, the Native American sweat lodge, the Russian bania, and the Central American temascal are all examples of the use of high temperature for personal cleansing and hygiene. The Greeks and Roman baths are similar with written records dating from the fifth century BCE. The popular explanation for the health effects of sauna-like treatments has been the “flushing” of toxins out of the body through increased sweating.

While modern medicine has had many advances in the understanding of human physiology, there is still a great deal that remains unexplained. Traditional medicines and practices are often a useful source of compounds and procedures to test for medicinal properties. Thus a method that improves the body's natural ability to regulate and enhance the formation, and release of nitric oxide may have significant and widespread health benefits.

Control and regulation of the generation and release of nitric oxide may provide a method to maintain proper blood pressure, vascular tone, coagulation properties of the blood and a host of other bodily functions. However, nitric oxide has a short lifetime in physiological fluids.

Nitric Oxide is a vasodilator and has also been implicated as a component of the human body's natural defense against disease causing organisms. Numerous disease causing organisms cause an increase in nitric oxide production of the body. Production of Nitric Oxide may be therapeutic, although too much nitric oxide is also implicated in some disease states.

Hemoglobin can reversibly bind nitric oxide to form S-nitrosohemoglobin. This compound forms in one part of the body and is transported by the blood to regions of reduced oxygen partial pressure where it decomposes releasing nitric oxide. The nitric oxide then causes dilatation of the capillaries where the oxygen content of the blood is low. This dilatation increases blood flow to those areas where it is needed most, those areas with reduced oxygen. A known source of S-nitrosohemoglobin is the lungs. Nitric oxide is produced in the nasal passages and is absorbed in the lungs improving the function of the lung by improving the match of blood and air flow. The nitric oxide also has effects on peripheral circulation.

Alzheimer's is believed to be a microvascular disorder with neurological degeneration secondary to hypoperfusion. Alzheimer's does not occur in all individuals, and it does not occur in single or even a few episodes of hypoperfusion, rather it occurs over time, sometimes over many years. The course of Alzheimer's, while inexorable and monotonic, is not steady, and is not associated with known episodes of hypoperfusion.

In the early stages there may be considerable variability in degree of neuropathy and in rate of decline making the diagnosis of Alzheimer's difficult in the early stages.

Nitric oxide induces a state in cells where those cells are more resistant to a following hypoxic or ischemic event by competitively nitrosylating mitochondrial caspases and inhibiting their activation during hypoxia and so inhibiting apoptosis.

Nitric oxide inhibits apoptosis downstream of cytochrome C release by nitrosylating caspase 9. NO inhibits mitochondrial cytochrome oxidase and so interrupts oxygen utilization by the mitochondria.

Nitric oxide may be produced by a variety of cells in the body under a variety of circumstances. Endogenous nitric oxide production is via nitric oxide synthase (NOS).

NOS comes in three isoforms, inducible (iNOS), endothelial (eNOS), and neuronal (nNOS). iNOS is Ca++ independent and can produce nitric oxide in the μM range but requires significant induction time. NO is also an antioxidant which rapidly destroys OH—. In Alzheimer's, oxidative damage is specific to RNA Oxidation is a prominent feature of vulnerable neurons in Alzheimer's disease. The oxidation products may be produced by hydroxyl from H₂O₂ and redox-active metals in the cytoplasm. Nitric oxide can scavenge superoxide, and H₂O₂. Thus the oxidation of RNA in the cytoplasm is consistent with oxidative stress in the absence of NO. Oxidized DNA was found to be absent.

NO may be produced in the nasal passages and absorbed in the lungs by hemoglobin in the high O₂ affinity R state and is carried by the blood to regions of reduced oxygen tension where after releasing O₂, deoxyhemoglobin in the T state releases NO. This release of NO causes vasodilatation in the flow determining arterioles and regulates flow by dilating the vessel in response to lowered O₂. However, the arterioles that regulate flow are necessarily upstream of the capillaries where oxygen release occurs, and in the presence of oxygen NO is oxidized to nitrite and nitrate by hemoglobin.

NO is readily taken up by deoxygenated hemoglobin (Hb) and is stable under anaerobic conditions in vitro. Hb has a greater affinity for NO than for oxygen but the presence of one NO on the oxygenated Hb tetromer (Hb-(O₂)₃NO) decreases the affinity for oxygen and so enhances the release of oxygen. The presence of hemoglobin enhances the diffusion rate of oxygen in whole blood. Nitric oxide further enhances the diffusion rate of O₂ because the rate limiting step in HbO₂ mediated O₂ diffusion is the dissociation of the Hb-O₂ ligand which is accelerated in Hb-(O₂)₃NO.

Hemoglobin is the most abundant protein in the blood, and is about 900 g in a 70 kg man. Basal NO derives from several sources, the majority is thought to be from eNOS. Release of NO from endothelia is stimulated by hydraulic shear produced by high velocity flow.

Severity of ischemia sufficient to produce levels of oxidative damage observed due to hypoperfusion would presumably produce noticeable contemporaneous mental effects. Levels of hypoxia and ischemia not producing oxidative damage are noticeable. Levels of hypoperfusion resulting in confusion or syncope are typically not reported, so the oxidative damage may have occurred during a non reportable time, and may have occurred during sleep.

During sleep, the metabolism of all parts of the body is reduced. The blood pressure falls and the blood flow decreases. The velocity of blood flow throughout the body decreases, and with less shear at the vessel walls eNOS is down regulated and NO production by eNOS is reduced. The energy demands of the brain are reduced. The brain however is still quite active and still requires substantial blood flow.

Hypothermia is known to reduce cerebral damage during ischemic events. Hypothermia both during and even after such events has been shown to reduce brain damage by reducing the reperfusion injury. Sleep normally causes a drop in body temperature of 0.5-0.7° C. Mild hypothermia during sleep would reduce energy needs of the brain and would reduce the ischemic threshold for damage. The basal metabolism rises approximately 14% for every 1° C. of fever, so the “normal” reduction of 0.5-0.7° C. is a reduction of 7 to 10%.

The reports of a “protective effect” associated with NSAIDs, may be do in part to their effects at lowering body temperature, reducing basal metabolism and so reducing the damage associated with a given level of ischemia.

The epidemiology of Alzheimer's is well studied in developed countries but much less so in underdeveloped countries. Reliable and consistent diagnosis across many patients, many physicians, and many cultures is difficult and perhaps fraught with error. Tables 1 and 2 show the incidence of Alzheimer's reported by Suh and Shah Review Article: A review of the epidemiological transition in dementia—cross-national comparisons of the indices related to Alzheimer's disease and vascular dementia, Acta Pyschiatr Scand 2001: 104: 4-11. Table 1 shows maximum and minimum average monthly temperatures and incidence of Alzheimer's Disease and Total Dementia for undeveloped cities. Table 2 shows maximum and minimum average monthly temperatures and incidence of Alzheimer's Disease and Total Dementia for developed cities.

TABLE 1

| | Date | | Average | Average | Prevalence | Prevalence |
|---|---|---|---|---|---|---|
| Undeveloped | of | Hottest | High | Low | Alzheimer's | Total |
| City | Study | month | Temperature | Temperature | Disease | Dementia |
| Beijing | 1987 | July | 87.4 | 70.9 | 0.4 | 0.8 |
| Shanghai | 1990 | July | 88.9 | 76.6 | 3 | 4.6 |
| Hong Kong | 1998 | July | 92.7 | 74.5 | 4 | 6.1 |
| Taiwan (Taipei) | 1998 | July | 90 | 77.9 | 2.3 | 4 |
| lbadan (Lagos) | 1997 | February | 91.8 | 75.4 | 1.1 | 1.4 |
| Kerala | 1998 | April | 93.6 | 71.2 | 1.4 | 3.4 |
| (Bangalore) | | | | | | |
| Tokyo | 1982 | August | 87.6 | 75.2 | 1.2 | 4.8 |
| Okinawa | 1995 | July | 88 | 79 | 3.1 | 6.7 |
| Hiroshima | 1999 | August | 87.6 | 74.5 | 2.9 | 7.2 |
| Aichi (Nagoya) | 1986 | August | 90 | 74.3 | 2.4 | 5.8 |
| Wuhan (Wuhu) | 1981 | July | 88.9 | 76.6 | 0.1 | 0.5 |

TABLE 2

| Un- | Date | | | | Prevalence | Prevalence |
|---|---|---|---|---|---|---|
| developed | of | Hottest | Average | Average | Alzheimer's | Total |
| City | Study | month | High | Low | Disease | Dementia |
| Beijing | 1999 | July | 87.4 | 70.9 | 4.8 | 7.8 |
| Boston | 1989 | July | 81.8 | 65.1 | 8.7 | 10.3 |
| Odense | 1997 | August | 69.4 | 52.2 | 4.7 | 7.1 |
| London | 1990 | July | 71.1 | 52.3 | 3.1 | 4.7 |
| Stockholm | 1991 | July | 71.4 | 56.1 | 6 | 11.9 |
| Rotterdam | 1995 | July | 85.5 | 43.7 | 4.5 | 6.3 |
| (Amster- | | | | | | |
| dam) | | | | | | |

Reported temperatures were taken from tabulated monthly averages from Yahoo weather, www.yahoo.com. When average monthly temperatures were not available, they were taken from a nearby city (in parentheses). The data was divided into two sets, a “developed” and an “undeveloped” group. Beijing was included in both, with 1987 data as “undeveloped” and 1999 data as “developed.” The two groups were divided on the basis of perceived per capita water consumption for bathing. The relevant population is the populations at risk for Alzheimer's which is likely to lag behind others in the adoption of new bathing practices.

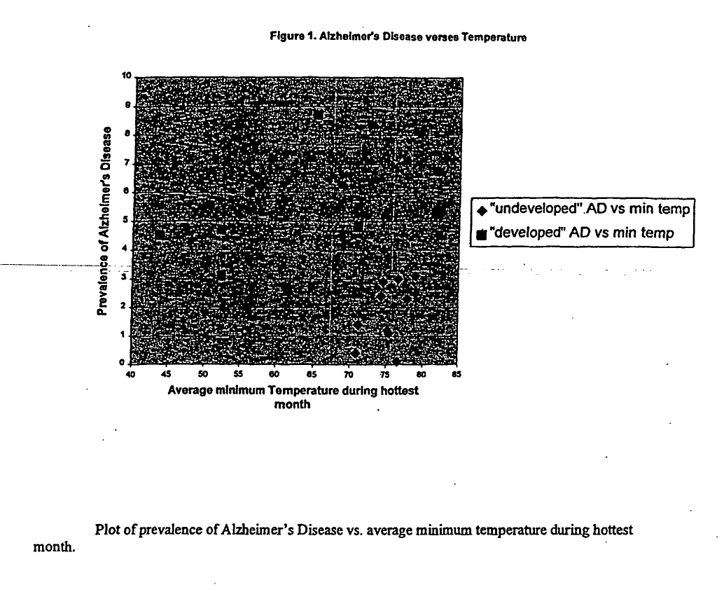

The data is plotted in FIG. 1. The two data sets fall into two groups, with increased minimum temperature correlating with increased incidence of Alzheimer's Disease, but with a different slope and intercept. The undeveloped intercept is about 70° F. Any intercept for the “developed” group would be off the chart, and would be unrealistic because heating would be used to raise the temperature into a “comfort zone”.

The lower incidence of Alzheimer's Disease in less developed regions may likely due to differences in bathing practices. When the head is unwashed, sweat residues accumulate on the scalp and serve as a growth media for autotrophic ammonia oxidizing bacteria. These bacteria generate nitrite and nitric oxide which can be absorbed into the skin where it is taken up by the blood in the scalp capillaries. Generation and absorption of nitric oxide in the terminal capillaries where oxygen tension is low, reduces the opportunity for nitric oxide destruction by oxygen.

This nitric oxide is then available during sleep in the blood to act upon the brain just prior to events of ischemia and to down regulate the mitochondria and so prevent ischemic damage to brain cells. Nitric oxide generated on the unwashed scalp is seen to be protective against ischemia so higher temperatures are tolerable without ischemic damage to the brain.

The advantage of applying nitric oxide producing bacteria to the scalp, is that the body has evolved to utilize such bacteria, and has also evolved physiological methods of controlling and utilizing that nitric oxide. Nitrate can be applied to the scalp and nitric oxide can be generated by heterotrophic bacteria and so prevent Alzheimer's. In a preferred embodiment, method ammonia oxidizing bacteria are applied to the scalp along with substrates for these bacteria to generate nitric oxide. These substrates include ammonia or urea as the electron source and nitrate or nitrite as the electron sink for bacterial metabolism.

Other methods of supplying nitric oxide to the body and brain may also be utilized to treat Alzheimer's Disease. These include supplying a nitric oxide donating molecule either orally, transdermal, by injection or by inhalation of a nitric oxide containing gas. By far the most convenient is the application of nitric oxide producing bacteria to the scalp. In that the nitric oxide is needed during sleep, a convenient time of application is prior to sleep although such material can be applied at any time and can accumulate in the blood as (Hb-(O₂)₃NO) and as various nitrosylated compounds.

Other sources of nitric oxide can also be used, nitric oxide donating compounds such compounds selected from the list of: nitric oxide, organic nitrates, inorganic nitrates, organic nitrites, inorganic nitrites, nitroglycerine, compounds containing a nitrosylated sulfhydryl group, erythrityl tetranitrate, pentaerythritol tetranitrate, isosorbide dinitrate, S-nitrosoglutathione, sodium nitroprusside, S-nitrosocysteine, S-nitrosocysteinylglycine, S-nitroso-(gama)-glutamyl cysteine, nitrosohemoglobin, S-nitroso-L-penicillamine, 7-nitrosoindazole, S-nitrosomemantine, L-arginine and mixtures thereof.

L-Arginine is an amino acid which is the normal substrate for the production in the body of nitric oxide by nitric oxide synthase. Supplementation with L-arginine can increase nitric oxide production through stimulation of production of nitric oxide by nitric oxide synthase. Autotrophic ammonia oxidizing bacteria utilize ammonia and generate nitrite, similarly some heterotrophic bacteria can utilize nitrate as the terminal electron sink and generate nitrite. Any compound that releases nitric oxide may be used to prevent Alzheimer's.

More specifically, applying a composition of an ammonia oxidizing bacteria to skin during or after bathing to metabolize urea and other components of perspiration into nitrite and ultimately into nitric oxide results in a natural source of NO. One aspect of the present invention causes topical nitric oxide release at or near the surface of the skin where it can diffuse into the skin and have local as well as systemic effects. This naturally produced nitric oxide may then participate in the normal metabolic pathways by which nitric oxide is utilized by the body. Adding urea or ammonium salts to the skin provides additional substrates that these bacteria utilize to form nitrite. As used herein, the phrase near the surface is defined as adjacent to or in close proximity to, and may, but need not be in contact with the surface.

The invention may be understood by understanding that until the advent of running hot water and soap, bathing was infrequent. Under such conditions (prevailing for >99.9% of historic and prehistoric time) the skin would develop a natural community of microorganisms adapted to the skin environment. An abundant component of human perspiration is urea. In soil, natural bacteria act upon urea and hydrolyze it to ammonia, which is then oxidized to nitrite, followed by rapid oxidation, by still other bacteria, to nitrate. In soil, all nitrogen containing compounds are ultimately degraded to nitrate. In fact it is nitrate that most plants absorb as their nitrogen source. Under conditions of infrequent bathing, skin bacteria that can metabolize urea into nitrite would thrive and proliferate. The resulting nitrite on the skin when dampened by additional perspiration at the normal sweat pH of 4.5 would release NO.

Bathing has as one of its primary objectives removing bacteria from the skin. While pathogenic bacteria are undesirable, all bacteria are not pathogenic. Recent advances in soap formulations have included the adding of broad-spectrum anti-microbial agents to soap. Bathing has greatly reduced the incidence of water-borne diseases such as cholera and various diarrhea diseases. It may be that removal of all bacteria has the undesired effect of removing the natural bacteria that produce nitrite, which the body has evolved to utilize physiologically.

In another embodiment of the invention, nitric oxide may be used to treat other diseases and disorders, such as obesity and Diabetes Type 2. Obesity, as used herein, is defined as a body mass index greater than or equal to 30 or about 30 pounds overweight for a 5′4″ person. According to The American Association of Clinical Endocrinologists, one in three Americans may be at higher risk for diabetes and coronary heart disease that has previously been attributed to excessive consumption of food and insufficient physical activity. At the same time however, many people are preoccupied with being thin, dieting, and exercise. Efforts to lose weight and to remain thin are much greater than the efforts that were made by previous generations, but to much less effect. The epidemic of obesity is occurring in spite of these efforts.

Much credence has been placed on the notion that people evolved under conditions that required more physical exertion than is necessary today, and that the present epidemic of obesity results from present levels of physical activity that are less than in prehistoric times. In contrast to this explanation caged animals rarely become morbidly obese the way that humans frequently do. Typically animals with abundant food supplies increase their reproductive rate. However the birth rate in the developed world is declining and is substantially lower than in the undeveloped world.

Accumulation of fat occurs whenever calories are ingested faster than they are used. As an evolutionary principle, the accumulation of fat can be seen as the storage of calories from current times of plenty to times of future potential scarcity. Such an evolutionary driving force should only reflect food availability, it should not necessarily be modulated by degree of physical activity necessary to obtain that food. If anything, a higher level of physical activity would imply a greater metabolic “cost” of obtaining food and a greater incentive for the accumulation of fat with physical activity, the opposite of what is observed.

In the developed world, food is cheap and widely available. However, while food scarcity has caused malnutrition at isolated times and in isolated regions, a lack of food does not seem to be a factor which prevented widespread obesity 50 years ago. The advent of television and computer based employment and entertainment has reduced physical activity, but only to a modest extent. Few tasks now or in the 1950's had a significant component of physical manual work. Sitting at a desk and writing by hand is only slightly more physically demanding than sitting at a computer and typing. The epidemic of obesity is occurring now, not in the 1960's or 1970's when television was introduced and became widespread.

There are two types of “diabetes.” The first, type 1, results from a destruction of the pancreatic islets which produce insulin, which results in a profound loss of the ability of the body to produce insulin. This insulin must then be supplied from external sources. Untreated type 1 diabetes can result in extremely high blood sugar as well as other health disturbances. The second type of diabetes, type 2, is characterized by a loss in sensitivity to insulin, which is somewhat compensated by increased insulin secretion and elevated blood sugar levels. There is a strong correlation between type 2 diabetes and obesity. Typically obese individuals lose sensitivity to insulin and become type 2 diabetic. To some extent the metabolic changes that occur with type 2 diabetes may be corrected through weight loss, diet, and exercise.

Surprisingly, I have found that Alzheimer's and the epidemic of obesity, diabetes, high blood pressure, and heart disease are all related, not to diet, but to the bathing practices of the developed World. Frequent bathing may wash off the previously unrecognized comensal autotrophic ammonia oxidizing bacteria that in the “wild” live on the skin and metabolize the urea in sweat into nitric oxide. I believe that it is the loss of this source of nitric oxide that, by reducing the basal levels of nitric oxide, force the physiology of the body to adjust, and to attempt to provide sufficient nitric oxide by utilizing other physiological pathways that are not well adapted for basal nitric oxide production. I believe that high blood pressure, Alzheimer's disease, obesity and type 2 diabetes are a consequence of these physiological changes. Nitric Oxide is a small molecule that diffuses rapidly through the skin into the capillaries of the skin. Vasodilatation of these capillaries would occur, as well as diffusion of NO into the blood where it may be transported to other regions of the body. Dilatation of the capillaries at the skin surface enhances blood flow to, and hence heat loss from, the skin during periods of exercise.

Nitric Oxide has been studied for treatment for chronic tension headaches, sickle cell anemia, impotence, tumors, and heart disease. Heart disease and other vascular diseases are a significant cause of death in the developed world. Vascular diseases also cause significant reductions in quality of life for those afflicted. Significant medical resources are devoted to prevention, treatment and research into the causes of these forms of disease.

The main previously known sources of nitric oxide are the various nitric oxide synthase enzymes (NOS) one of which, nNOS, may be the most structurally diverse human gene described to date. Lesser amounts of nitric oxide are produced by reduction of salivary and dietary nitrate by heterotrophic bacteria on the tongue and in the gut. Nitrate in the diet has been thought to be detrimental, primarily due to the possibility of methemoglobin in infants, the so called “blue baby syndrome.” Some of the protective effect of a vegetarian diet may well be due to the high levels of nitrate in green leafy vegetables being reduced by bacteria in the gut resulting in higher basal nitric oxide levels.

Exercise does increase basal nitric oxide levels through the stimulation of eNOS, which may mediate the protective health effects of exercise. The statins (HMG-CoA reductase inhibitors) reduce stroke damage, and that some of that protective effect is mediated through the up-regulation of eNOS. Long term (1 month) oral L-arginine (the substrate for NOS) increases insulin sensitivity in type 2 diabetics while also increasing cGMP (the product of guanylyl cyclase when stimulated by NO) while increasing peripheral blood flow while reducing peripheral resistance. Oral L-arginine also improves endothelium dependent dilatation in hypercholesterolemic young adults. In healthy normotensive individuals, insulin sensitivity and eNOS activity are positively correlated.

Chronic (8 week) inhibition of NOS with L-NNA increases serum triglyceride and body fat and reduces serum nitrate (end metabolite of NO) in rats where dietary supplementation with L-arginine suppresses the elevation in body fat and the reduction in serum nitrate. Nitric oxide increases glucose transport in rat skeletal muscle, and this increased glucose transport is additive to that of insulin at physiological concentrations. Myocardial glucose uptake is regulated by NO via eNOS where NO donors shut off glucose uptake. Inhibition of NOS increases cardiac glucose uptake and reduces free fatty acid uptake. Inhibition of NOS switches cardiac metabolism from fatty acid to lactate and glucose utilization. This switching is reversed by a NO donor.

Insulin stimulates the release of nitric oxide in human umbilical vein endothelial cells, in part through the phosphatidylinositol 3-kinase (PI 3-kinase) pathway. Blockage of NO synthase increases NO synthesis and increases insulin release from isolated mouse islets. Insulin reduces reperfusion injury of cardiomyocytes, and retinal neurons, through inhibition of apoptosis mediated through the P13-kinase. Nitric oxide inhibits glucose induced insulin secretion from pancreatic islets. Nitric oxide may be a link between insulin resistance and cardiovascular morbidity.

Thus many of the metabolic disorders observed in obese and type 2 diabetic individuals, hypertension, elevated serum lipids, insulin resistance, elevated insulin are made worse by the inhibition of NOS, and are made better by increased NO either through L-arginine or through NO donors or through exercise, or through a vegetarian diet (which happens to be rich in nitrates).

In one embodiment of the invention, basal nitric oxide level is increased by applying autotrophic ammonia oxidizing bacteria to the surface of a subject, such as skin or scalp.

In one embodiment of the invention urea in sweat is utilized to form nitrite through ammonia and urea oxidizing bacteria. Nitrate in the diet is rapidly absorbed and is concentrated by the body in the saliva. In the mouth facultatively anaerobic bacteria on the tongue metabolize nitrate to form nitrite. Saliva contains significant nitrite and studies have shown that when the skin is licked that NO is released. This NO is believed to have anti-microbial and vasodilator effects. The release of NO is the rationalization as to why animals (and humans) lick wounds to enhance healing. Similarly a common folk remedy for impotence is the use of saliva directly applied to the penis where NO release would induce and prolong erection. Salivary nitrite may reduce food born illness, in that chloride present in the stomach in a high concentration will catalyze nitrosation reactions to form additional reactive intermediates that may add to the toxicity of acidified nitrite. On the skin, the concentration of chloride can reach that of a saturated salt solution, levels much higher than can be reached in the stomach.

Nitric oxide regulates mitochondria respiration by inhibiting cytochrome oxidase in competition with oxygen. The degree of inhibition is dependant on the oxygen concentration, and this inhibition increases at lower oxygen concentrations. The partial pressure of oxygen falls with altitude, so for constant basal nitric oxide levels, at higher altitude, nitric oxide exhibits a greater inhibitory effect.

The level of basal nitric oxide necessary to inhibit and regulate the consumption of oxygen by cytochrome oxidase will be lower at lower levels of ambient oxygen, and so diseases associated with insufficient basal nitric oxide may show reduced incidence at higher elevations.

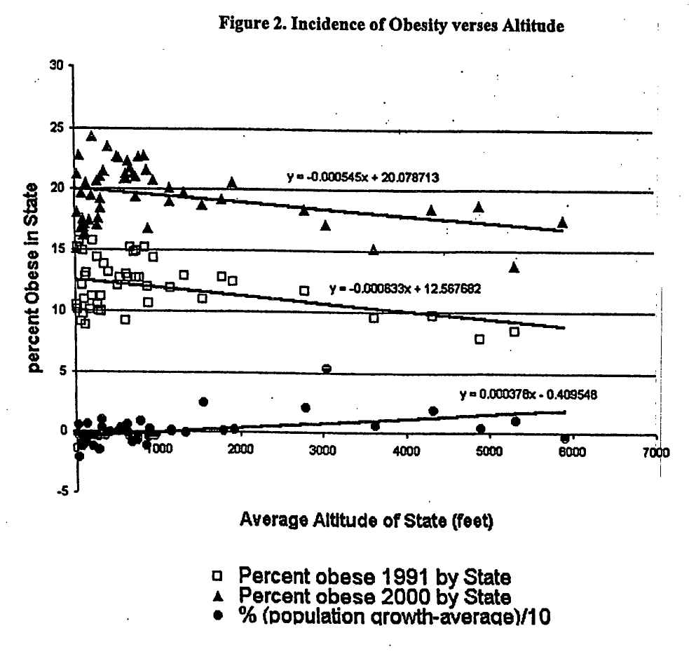

In FIG. 2 is plotted the incidence of obesity in the US population for 1991 and 2000 by State from the CDC Behavioral Risk Factor Surveillance System (BRFSS) verses average altitude for that State. Average altitude was calculated by summing altitudes for population centers weighted by population.

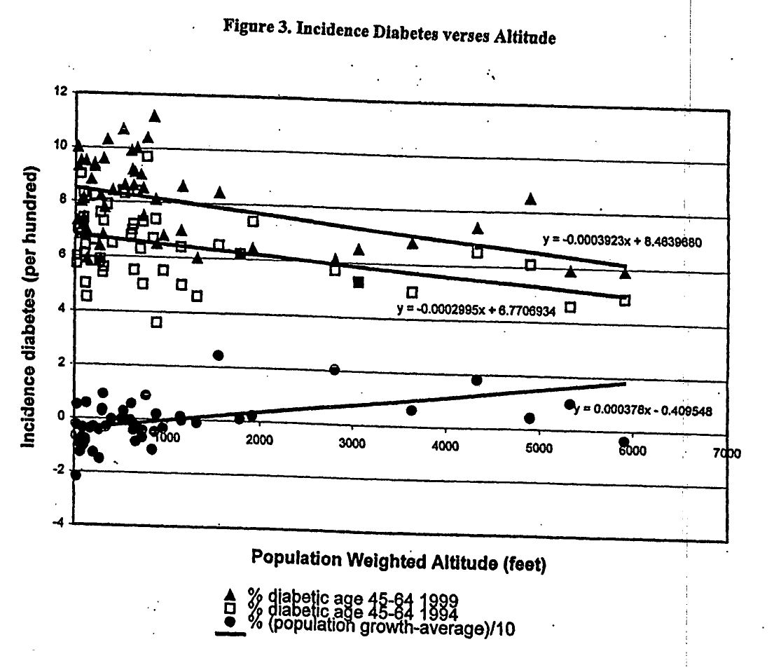

In FIG. 3 is plotted the incidence of diabetes in the US population in 1994 and 1999 by States verses average altitude in that State.

It can be seen that there is a trend for lower incidence of both diabetes and obesity at higher altitude. The highest incidence is at low altitude and all of the high altitude values are lower than the average. Also, obesity and diabetes take considerable time to develop, and there is significant population movement between low and high altitudes. In particular, the states with high elevations have been growing at rates that are faster than many states at low elevations. The population change (after subtracting the average growth from 1990 to 1999) is also plotted by state. It can be seen that the high elevation states grew faster than the average. Presumably, this excess growth resulted from people who lived at low elevations moving to high elevations. Obesity and diabetes are slow to develop, and so any effects of high altitude on obesity and diabetes may likely lag any change of address and may artificially skew the incidence at high elevations in the absence of population movement. An alternate explanation for the increased population growth at higher elevations, that of increased fecundity at higher altitude would also be consistent with a NO explanation.

Lower mortality from heart disease has also been observed in populations living at high altitude, and there is a positive correlation between living altitude and high density lipoprotein cholesterol. Thus, individuals living at higher elevations thus show reduced incidence of obesity, diabetes, heart disease and a marker for cardiovascular risk.

An important effect of the inhibition of cytochrome oxidase by NO, relates to the decoupling of mitochondria respiration rate from oxygen concentration. There are significant gradients in oxygen concentration between where oxygen is absorbed from the atmosphere and where oxygen is consumed in the peripheral tissues. Oxygen is not actively transported, but passively diffuses along a concentration gradient. Thus the sites of greatest oxygen consumption also have the largest gradients in oxygen concentration. Without a regulatory mechanism, the mitochondria closest to the oxygen source would consume more, and mitochondria farther away would get less or even nothing. This regulation of mitochondria oxygen consumption is especially important in tissues that consume large and variable quantities of oxygen. The delivery of oxygen by the blood is not continuous. Blood consists of about 40% red blood cells and about 60% plasma. It is the cells that carry oxygen. The heart moves blood by beating, so blood is moved in a pulsatile manner. Even if the blood were homogeneous and isotropic, the pulsatile movement of blood would result in a time varying delivery of oxygen. The oxygen consumption of heart muscle can vary by a factor of 10. The concentration of oxygen in the blood stays relatively constant, more oxygen is supplied by increasing the blood flow. However, the diffusion of oxygen from the blood to the cells can only be increased by increasing the gradient. Cells that are in the path of this gradient must regulate their oxygen consumption independent of the oxygen concentration.

The mitochondria respiration chain consists of a number of enzymes. The pathways by which the requisite levels of each active enzyme are regulated are not fully understood. There may be short term (seconds) as well as longer term (hours) regulation and very long term regulation of days or weeks. The longer term regulation, may relate to gene expression, and the numbers of active enzymes of each type that are produced and are present on the mitochondria, and perhaps even the number of mitochondria. The short term regulation, may have a time constant comparable to the time constant of the variability with which it deals. The inhibition of cytochrome oxidase by nitric oxide has been shown to be part of that regulatory pathway. Efficient regulation of the respiration chain requires regulation at more than one point. Those regulatory pathways must be linked in order to work in concert. Each mitochondria (each cell can have thousands) must be regulated independently. Efficient respiration requires that the electron flow in the respiration chain be matched. An excess of any particular enzyme, allows the reduced form of that enzyme to accumulate and in the presence of O₂, form super oxide.

Many of the modern chronic diseases, heart disease, diabetes, hypertension, stroke, Alzheimer's Disease and even cancer, are thought to be related to the production of free radicals and the oxidative damage that such reactive oxygen (ROS) and reactive nitrogen species (RNS) cause. However, supplementation of the diet with anti-oxidant vitamins E and C and beta carotene apparently to not produce any significant reductions in the 5-year mortality from, or incidence of, any type of vascular disease, cancer, or other major outcome.

It is becoming apparent that ROS and RNS are normal products of metabolism, and are used as messenger molecules. I believe that the lack of sufficient nitric oxide, releases the inhibition of cytochrome oxidase and allows that enzyme to operate at higher than normal rates. The result is a higher instantaneous consumption rate of oxygen. The “average” oxygen consumption remains the same because total oxygen consumption remains the same, therefore the variation in oxygen consumption with time increases. When the blood flows in a pulsatile manner, the oxygen delivery to the wall of the capillary is also pulsatile. Oxygen will diffuse toward the mitochondria and be consumed there. The oxygen delivery to the mitochondria will necessarily be pulsatile also. With a higher instantaneous rate of oxygen consumption, the difference between the maximum O2 concentration and the minimum O2 concentration will increase, particularly far from the vessel wall. NO inhibits cytochrome oxidase by competing with O2. As O2 levels drop, the inhibition increases and the consumption rate falls. With sufficient NO, this regulation of O2 consumption allows for O2 to reach cells that are farther away from the vessel wall. With less NO, the rate is faster at high O2 levels, and much faster (relatively) at lower O2 levels. Thus the O2 level can drop to where the mitochondria respiration chain becomes fully reduced. It is not the absolute level of O2 that causes the production of superoxide, but rather changing from anoxic to oxic. I suspect that a portion of the lumen wall thickening that is often observed in heart disease may be adaptive in the sense that by providing more resistance to O2 diffusion, it reduces the O2 concentration change between oxic and anoxic conditions, and so reduces superoxide formation during time varying O2 delivery or consumption.

Superoxide is produced when O2 picks up an electron from a reduced enzyme other than cytochrome oxidase. The electron that is shuttled to the cytochrome oxidase comes from either NADH dehydrogenase or succinate dehydrogenase. I suggest that with insufficient basal nitric oxide, the mitochondria become more sensitive to fluctuations in O2 demand, and that the production of superoxide is unavoidably increased.

Virtually all important metabolic systems are under some type of feedback control. Nitric oxide is involved in many feedback control loops, including the regulation of peripheral vascular resistance by shear stress dependant NO release followed by vessel dilatation. It would be surprising if basal nitric oxide were not under feedback control as well. A difficulty with the feedback control of nitric oxide, is that it diffuses readily, and it has a short half life. A source of NO must produce an NO concentration higher than the sink which consumes it. Nitric oxide is toxic at high levels, and any source of nitric oxide should be regulated, either in time, by feedback, or in space. If basal NO concentration is regulated by feedback, inhibition of some sources will cause other sources to be up-regulated.

For example, the hypotension of septic shock is thought to be largely from the excess production of nitric oxide by iNOS. iNOS is the inducible form of NOS, and is an example of a “feed forward” type of control, rather than a “feed back” kind of control as in eNOS. The production of very high levels of nitric oxide by cells is best achieved by a “feed forward” type of control. Once a cell starts to produce high levels of nitric oxide, the nitric oxide so produced will inhibit the cytochrome oxidase of the mitochondria in those cells and will interfere with normal cell metabolism.

Production of basal nitric oxide by human granulocytes has been shown to be time periodic, with a period of a few minutes, and in the 1000 pM range. These measurements were done 10 μm above a pellet of 10E3 cells, This periodic signal was necessarily an average from many cells. That a periodic signal was observed indicates that the cells were producing NO at a time varying rate, and that this NO production was in phase. Maintaining phase coherence over so many cells, would indicate communication between cells, and feedback. It is possible that some other messenger molecule mediates the communication between cells, however any such molecule would need to have a shorter lifetime than NO in order to maintain phase coherence. The most plausible explanation is that there is direct sensing of nitric oxide concentration, and feedback regulation of nitric oxide production, albeit with a time lag.

Assuming basal NO is subject to feedback control, the basal NO level set point is below where adverse health effects from insufficient NO are apparent. It may be that since nitric oxide is produced in response to physical activity, humans evolved to rely upon the nitric oxide produced by the moderate physical activity needed for a hunter-gatherer lifestyle, where “daily” physical activity levels produced sufficient nitric oxide, and so there was no evolutionary pressure to evolve other nitric oxide sources. However, the inventor believes that humans relied on another previously unrecognized source of nitric oxide during prehistory, that of the comensal autotrophic ammonia oxidizing bacteria, and that the frequent bathing of a modern lifestyle removes this source of nitric oxide.

Modern dietary practices and lack of physical activity may not be a cause for obesity and diabetes, but that modern bathing practices have caused a reduction in basal nitric oxide may be a cause.

I have found that specific strains of these common bacteria can live on the scalp and produce nitric oxide and that this bacterially produced nitric oxide has important and beneficial health effects. These are the autotrophic ammonia oxidizing bacteria. They derive energy solely from the oxidation of ammonia to nitrite or to nitric oxide. They derive organic carbon almost entirely by fixing CO₂. They are slow growing, with optimum doubling times of 10 hours compared to 20 minutes for heterotrophic bacteria. These bacteria are ubiquitous in soil, where they are responsible for metabolizing ammonia released into the soil into nitrite which is then oxidized by another type of bacteria into nitrate in the process of nitrification. These bacteria are important in soil chemistry and in waste water treatment. There has been no known reported case of infection due to these bacteria, or even any prior association with the human body. The lack of association of these bacteria with the human body may be due to the fact that these bacteria do not grow on the standard culture media that are used for isolating heterotrophic bacteria and pathogens, and as slow growing bacteria, it is easy to wash them off the skin faster than they can proliferate. Bathing even a few times a week would reduce populations to levels where isolation would be difficult. In the developed world (where most testing is done) bathing is in general frequent enough to eliminate any natural population of these bacteria, which in any case, would not be recovered in a test for heterotrophic bacteria (all known pathogenic bacteria are heterotrophic).

It is likely that these bacteria are incapable of causing infection, even in immuno-compromised individuals. The substrates they require for growth, oxygen, ammonia, inorganic mineral salts are not available except on the external skin and other regions in direct contact with external air. Application to the external skin has resulted in suppression of body-odor causing heterotrophic bacteria and resolution of fungal (athlete's foot) and viral (plantar wart) infection.

Applying nitric oxide producing bacteria to the skin allows the administration of nitric oxide continuously for long periods. I have had these autotrophic ammonia oxidizing bacteria living on my scalp and subsisting solely on sweat residues for over 8 months.

Apparently these are natural comensal bacteria and that much of human physiology has evolved to better utilize these bacteria as a natural source of nitric oxide. Adrenergic sweating, by administrating urea to these bacteria, causes the prompt release of nitric oxide providing a rational explanation of the reason for sweating in response to hypovolemic shock. Controlling nitric oxide production and absorption through sweating allows the regulatory systems (sweat glands) to be remote from the site of nitric oxide production. The use of autotrophic ammonia oxidizing bacteria to generate nitric oxide allows for very high local levels. Autotrophic bacteria can produce levels of nitric oxide that would be toxic to other types of cells. Cultures of these bacteria recovered from my scalp easily produce nitrite concentrations in the millimolar range. Evaporative concentration could produce even higher concentrations on the skin. Nitric oxide is used to cure meat and prevent its spoilage by bacteria. Curing the outer dead layers of skin with bacterially generated NO may make the skin very resistant to topical infection.

With this in mind a number of curious aspects of human physiology can be understood. The areas of the body that are most in need of rapid healing, infection control and hence of NO production are feet, hands, scalp, and genital area, which are the parts of the body where perspiration is most abundant even when not needed for cooling. This may be why there is urea, chloride, and iron in perspiration, and why perspiration has a low pH.

Sauna and other types of sweat baths can be seen as ways of enhancing the production of nitrite and NO on the skin. Modern use of the sauna as part of a bathing ritual involving washing with soap and running water would not achieve such a result and has only been practiced since the 19^{th} century. Urea and nitrite are very water soluble and would be washed off readily. When the custom of sauna first developed over 2000 years ago, there was no running hot water, so the skin would retain the soluble urea and nitrite, and without soap the bacteria would also be retained.

Another custom, the use of a whisk, a bundle of birch branches used to gently beat the skin, may be seen as a method of inoculating the skin with bacteria present on the whisk. Between uses the whisk would dry out and the bacteria surviving would necessarily become adapted to living on perspiration residues under fairly dry conditions, the natural state of human skin. The advent of the germ theory and the perceived need for aseptic hygienic conditions has modified the use of such devices to where they probably no longer serve this original function.

The beneficial health effects of sweating can be seen as deriving from the increased production and release of NO on the skin, rather than due to removal of wastes. Perspiration as a waste removal method would seem to be a non-intuitive and ineffective method. Most sweating occurs during periods of high metabolic load, which would seem to be an inopportune time to use any metabolic capacity to rid the body of waste products. In fact the kidneys shut down under conditions of insufficient heart output. Sweat output can vary greatly from day to day, hour to hour, and minute to minute. Accumulating wastes in anticipation of episodic sweating events would seem to be a poor allocation of resources. Nervous sweating that occurs in anticipation of stressful events may be the body's way or preparing itself for a stressful event. Dampening the skin with perspiration would release NO from the newly generated and accumulated nitrite that would then act as a vasodilator, which may enhance blood flow and prepare the body to respond effectively to the stressor. Organic nitrates like nitroglycerine are often prescribed for exactly such use prior to physical or emotional stress to achieve just such vasodilatation.

Sweating can then be seen as the solution to skin disorders, rather than as the cause. If the ammonia and urea oxidizing bacteria content of the skin were restored to pre-industrial levels, then areas of the body with profuse sweating would also have profuse nitrite and NO production and would be expected to heal faster, better resist infection, and be in a better general state of health. In the absence of such bacteria, which happen to be relatively slow growing, other faster growing heterotrophic bacteria would hydrolyze urea to free ammonia which is quite toxic and irritating to the skin. It is thus the absence of the proper bacteria that cause perspiration residues to become irritating. Oxidation of ammonia to nitrite or nitrate lowers the pH, and converts any remaining free ammonia to the less toxic ammonium ion.

After formation on the skin, nitric oxide may diffuse into the capillaries of the skin and be taken up by the blood. The capillaries of the skin may dilate in response, and some of the nitric oxide may be taken up by hemoglobin to form S-nitrosohemoglobin which will circulate though out the body and have systemic effects.

A region of the skin that is thin and has abundant blood capillaries is the skin of the head and scalp. The presence of hair on the head is often rationalized as limiting heat loss. But one wonders why the scalp has hair to prevent heat loss while the rest of the body remains essentially hairless. There is significant heat loss from the head, possibly a result of the skin of the scalp being thin so as to allow the rapid diffusion of nitric oxide into the blood. The blood from the scalp joins that from the brain before entering the heart and lungs. The blood supply of the brain is among the most critical to the body and shows little variation even during periods of extreme metabolic stress. Combining blood having low oxygen tension from the brain with blood having a high nitric oxide content from the scalp may be an efficient use of the nitric oxide so produced. Because nitric oxide may also be released into the air around the head and face, where air drawn in during breathing in close proximity to a source of nitric oxide, the concentration of nitric oxide would increase in inspired air. The different patterns of facial hair seen between men and women may derive from different patterns of peak metabolic activity, men during hunting and fighting and women during pregnancy, labor and childbirth. Just as inhaled nitric oxide is protective of pulmonary hemorrhage for horses, nitric oxide released by bacteria on facial hair may facilitate greater levels of physical exertion.

Many of the veins of the scalp drain through the skull into the vascular sinuses in the brain. The arteries bringing blood to the brain pass through these same sinuses. This contact may be so that nitric oxide may diffuse from the venous blood leaving the scalp into the arterial blood entering the brain. Such diffusion would help explain the protective effect of moderate exercise on stroke. Sweat on a head may then reduce the vascular resistance in the brain.

Additional benefits of an embodiment of the invention may be seen in the following non limiting example.

### EXAMPLE I

An adult male subject with moderate male pattern baldness (estimate ⅓ loss) and long curly hair (0.2 m length) applied a barnyard soil derived culture enriched in ammonia oxidizing bacteria to his scalp. Blood pressure was recorded upon arising each morning for a total of 27 days, including a pretreatment period of 11 days, a treatment period of 5 days and a post treatment period of 11 days. The culture was applied each evening during the treatment period, and the hair was not washed during the treatment period. Systolic values are displayed in FIG. 1, wherein the diamonds depict the blood pressure readings during the pretreatment period, the circles depict the readings during the treatment period, and the x's depict the reading during the post treatment period. The triangles do not represent blood pressure readings, but merely the time of culture application. The systolic values averaged 9 mmHg lower during the daily application period while diastolic values (not shown) averaged 2 mmHg lower. Subjective effects included very slight headache for the first few days after application and transiently during episodes of increased sweating, increased tolerance toward elevated ambient temperatures, increased mental acuity, increased calmness similar to the effects of exercise, a pronounced darkening of the area around and under the eyes and an increased prominence of the veins of the forehead.

The culture was prepared by the following non-limiting method. Production of enrichment culture: From a local farm, soil was collected from separate pens containing pigs, sheep, horses, and cows. A nutrient solution containing ammonia was added along with ground limestone (as buffer) to each sample and the mixtures allowed to stand with occasional mixing. Nitrite production was monitored. Five samples including one from each animal pen, exceeded 5 mM NO2—. These samples were combined (after decanting residual buffer) and formed a first enrichment culture. A second enrichment culture was obtained from the first by incubating at 32° C. with nutrient solution at 2× concentration. The 2× culture was incubated at ambient conditions with added 1× nutrient solution and decanted several times. Approximately 1 ml of flocculated biomass in 13 ml of nutrient solution was applied to the scalp each evening for 5 days.

The solutions were designed to simulate the inorganic composition of human sweat. The solution consisted of 1.5 g NaCl, 0. 0.55 g KCl, 0.25 g CaCl₂, 0.24 g MgSO₄.7H₂O, 0.02 g K₂HPO₄, 2.0 g NaHCO₃, 1.5 g NH₄Cl in 1.0 l Milli Q+water (Millipore Corporation, Bedford, Mass.). Trace minerals (6 g FeSO4.7H₂0, 3.5 g CuSO₄.5H₂O, 0.25 g MnSO₄.4H₂O, 0.03 g Co(C₂H₃O₂)₂.4H₂O, 5 g ZnSO₄.7H₂O, 0.125 g Na₂MoO₄.2H₂O, 0.015 g KI, 2 g EDTA, in 1 liter) were added (1 ml/l of solution) to both solutions to achieve levels reported in sweat. Co and Mo levels in sweat were unknown but were added to achieve concentrations similar to those in published growth media for ammonia oxidizing bacteria as in Watson, S. W., Valois, F. W., and Waterbury, J. B. The Family Nitrobacteraceae, in The Prokaryotes Volume 1. (Starr, M. P., et al. eds.), pp 1005-1022, (Springer-Verlag, Berlin, 1981) incorporated herein by reference. All other components were within reported ranges for human sweat as published in Diem, K. and Lentner, C., ed., Scientific Tables, seventh edition, Ciba-Geigy Limited, Basle, Switzerland (1970) incorporated herein by reference. Interestingly, ¼ of ingested iron is reported to be excreted in the sweat. The solution was prepared by adding the dry salts to autoclaved Milli-Q+water immediately prior to use. The pH range for sweat is 4 to 6.8. In this pH range most ammonia is present as ammonium ion which is unavailable to the ammonia oxidizing bacteria. Urea should be available even at low pH. Earlier studies demonstrated that the urea is rapidly (hours) hydrolyzed to ammonia, so ammonium chloride and pH 7.8 was used to reduce the need for pH adjustment and to provide for ammonia availability. Bicarbonate was chosen as the major non-chloride anion instead of lactate to reduce the possible growth of heterotrophic bacteria.

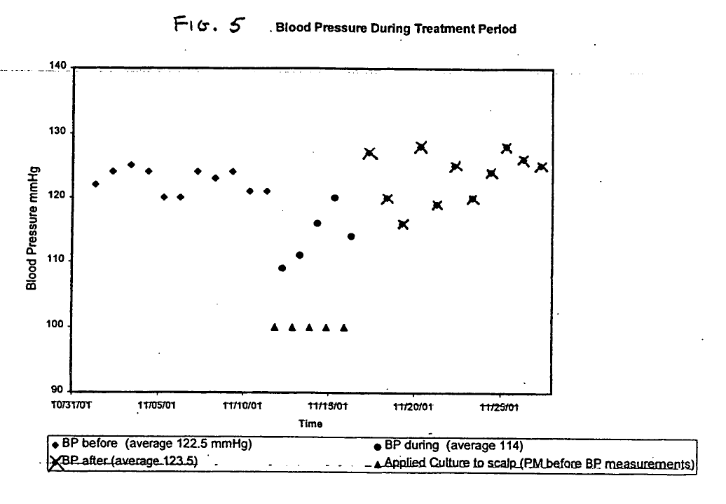

As seen in FIG. 5, the systolic blood pressure averaged 122.5 mmHg before and 123.5 mmHg after the treatment period, while the systolic blood pressure averaged 114 mmHg during the treatment period. Thus the lowering of blood pressure is demonstrated by the application of ammonia oxidizing bacteria to the scalp of a subject.

Hair can be seen as an insulating material but also as a surface on which ammonia oxidizing bacteria may proliferate. Hair may also be an absorbent material to prevent sweat from dripping off and may also provide a suitable micro-climate for the ammonia oxidizing bacteria. Nervous sweat is mediated through the adrenergic pathway and typically stimulates sweat on the head and neck.

Sweating for non-cooling purposes can be seen as a natural way for the body to increase nitric oxide production. The malaria parasite is sensitive to nitric oxide, particularly under conditions of low oxygen tension, as in venous capillaries. When nitric oxide synthase inhibitors are given to animals with malaria, the mortality is increased. The excessive sweating that is one of the symptoms of malaria (and of many other infections) can be seen as one of the body's natural nitric oxide increasing mechanism.

Human sweat has a high concentration of lactic acid. This renders the normal pH of sweat in the 4.5 range, where nitrite rapidly decomposes to release NO. Sweat also contains abundant iron. A quarter of ingested iron is excreted in the sweat. Iron is well known to coordinate with NO and form iron-nitrosyl complexes. Nitric oxide also reacts with superoxide to form peroxynitrite. In aqueous solution iron catalyzes reactions of peroxynitrite with other compounds to form toxic products. The presence of abundant iron and nitrite on the skin may be the first line of defense against skin infections.

Because the outer layers of skin are non-living, pickling and curing of these dead layers with nitric oxide prior to their sloughing off may have no detrimental health effects. Indeed, depending on bacteria for nitrite production and hence NO production, no living part of the body need be exposed to high levels of nitrite, nitric oxide, or toxic NO reaction products. Levels can be reached on the skin what would be detrimental to living tissues. This may be a very effective way of warding off skin infections, and may be the system that humans have evolved to utilize in the absence of frequent bathing.

The effect of exercise on vascular disease may also be seen in a new light. Moderate activity is both exercise and a way of, through inducing perspiration, increasing NO production. Vigorous activity does these things also, but if the sweating is sufficiently profuse, bathing is generally done after the exercise. Washing away the perspiration removes the protective effect of this NO production. This explains the reduced protective effect of vigorous exercise when compared with moderate exercise seen in some studies. The protective effect of exercise is increased but the protective effect of NO is reduced by bathing. That may indicate that the beneficial health effect of skin bacteria derived NO is, at least in some people, of comparable magnitude to that of exercise.

Reduced incidence of heart disease in summer may be due to the increased amount and time that sweat stays on the skin. In that the ammonia oxidizing bacteria are slow growing, they can easily be washed off faster than they can proliferate. That an annual pattern can be seen in the incidence of heart disease is strong evidence that this effect may be substantial.

Nitrite can be generated in several ways by bacteria. The first is by the oxidation of ammonia or urea. This is the necessary first step in the nitrification of ammonia in soil. Specific autotrophic bacteria utilize this ammonia oxidation to provide all their metabolic energy needs. A second type of autotrophic bacteria utilize this nitrite and further oxidize it to nitrate and utilize this energy for their metabolism. Other bacteria including heterotrophic bacteria can utilize nitrate to oxidize other compounds while reducing the nitrate to nitrite. At or below pH 5.5 nitrite decomposes releasing NO.

The reduced incidence of heart disease may be due to nitric oxide derived from the bacterial reduction of nitrate or to the bacterial generation of nitrite from ammonia or urea. Autotrophic bacteria may proliferate if there is a long interval between bathing, weeks or more, so that much of the observed differences in incidence is likely due to nitric oxide derived from sweat nitrate. However, the use of ammonia oxidizing bacteria in the present invention would allow for higher levels and larger effects on vascular health.

In the environment, ammonia and urea are oxidized to nitrite by Nitrosomonas, Nitrosococcus, Nitrosospira, Nitrosocystis, Nitrosolobus, and Nitrosovibrio. These bacteria are all lithotrophic Gram-negative bacteria that utilize carbon dioxide as their major carbon source. In the environment nitrite is oxidized to nitrate by Nitrobacter and Nitrocystis. Nitrosomonas is the most abundant of these types in soil and would be expected to be the most abundant on normal skin. These bacteria are autotrophs, that is, they do not utilize organic carbon for energy although some can assimilate organic carbon to a limited extent which can stimulate growth. All metabolic energy is obtained from the oxidation of the nitrogen containing species. The majority of carbon derives from the fixing of carbon dioxide utilizing this energy. Because these bacteria need only ammonia, oxygen, inorganic minerals, and carbon dioxide, they are expected to be completely non-pathogenic. The only part of the body where all of these are available is the exterior of the skin. They are slow growing when compared to other bacteria. Where E. coli has an optimum doubling time of 20 minutes, Nitrosomonas has an optimum doubling time of 10 hours. Because they do not utilize glucose or other organic compounds, they are difficult to culture and do not grow on the standard media used for isolating pathogens, which do utilize organic substrates for energy and growth. Some strains can also utilize urea directly.

Administering nitric oxide to the body in controlled amounts may have a number of beneficial health effects. It may reduce hypertension while increasing blood flow, increasing heart efficiency and reducing heart load. It may reduce infarct size during both heart attack and stroke and it may increase sexual function for both men and women. Nitric oxide is a powerful anti-microbial agent which is active against viruses, bacteria, yeast and fungi. On external skin, nitric oxide may reduce heterotrophic odor causing bacteria as well as skin infections.

In that the body has evolved to utilize these bacteria, there are a number of natural physiological regulatory mechanisms to control nitric oxide production and absorption. The production of nitric oxide begins with the release of urea through sweating to the bacteria which are in a biofilm on the external skin. Ammonia oxidation is inhibited at low water activities. As the nitric oxide is produced, it must diffuse from the source to the capillaries in the scalp. The diffusion resistance of the biofilm depends strongly on whether the void fraction is filled with air or sweat. Nitric oxide can diffuse toward the scalp, or it can diffuse into the open air and be lost (or inhaled). As the biofilm fills with sweat, the rate of production of nitric oxide increases continuously as more sweat is released.

As the nitric oxide diffuses into the capillaries, the perfusion of the capillaries depends on the level of nitric oxide present. At high NO levels, the capillaries are fully dilated and the blood flow exceeds that which is required for the scalp metabolism. As a result, the capillaries are filled with oxygenated blood. The lifetime of nitric oxide in blood is a strong function of the degree of oxygenation. In deoxygenated blood, nitric oxide is stable for long periods on the order of hours. NO attaches to the alpha heme to form α-nitrosyl hemoglobin (αNOHb) and in intact, oxygenated erythrocytes, oxidizes to metHb with a half life of 21 minutes. In oxygenated blood NO is rapidly oxidized on the order of seconds. Thus the absorption and subsequent transport of nitric oxide from the scalp into veins is regulated as noted above, the veins from the scalp drain through the skull and into the venous sinuses in the brain, the cavernous sinus. Veins are also dilated by nitric oxide, and at high NO concentrations, the volume of these venous sinuses would increase also increasing the time delay between when blood flows past the nitric oxide source on the scalp and when the blood can then deliver the nitric oxide to the arteries which pass through the venous sinuses, or to general circulation. When the venous blood passes through the lung, excess nitric oxide may be destroyed by the oxidizing conditions there. The effect of excessive NO is generalized vasodilatation. Were such vasodilatation to occur, it would divert blood flow away from the scalp, and so would reduce nitric oxide absorption.

The transport of NO by the blood is complex. NO is known to form multiple species including NOHb, S-nitrosyl hemoglobin, S-nitrosoglutathione, nitrite, nitrate and various S-nitrosoalbumins of different molecular weight. There is some thought that the binding of NO to NOHb is so strong so as to preclude the release of this NO at physiologically active levels. However, measurements of NOHb in vivo have demonstrated significant reductions in NOHb concentrations between arterial and venous blood during NO breathing. Vasodilation is reported even if less than 1% of observed artery-to-vein gradients in nitrosyl(heme)-hemoglobin during NO breathing (176, 468, and 340 nM NO at baseline, L-NMMA and exercise, respectively) were released and escaped reaction with oxygen and oxyhemoglobin.

Thus there may be multiple natural regulatory mechanism that may serve to limit the amount of nitric oxide that can be absorbed from the scalp and how much will reach the target tissues. In that there has been no reported case of nitric oxide “poisoning” due to ammonia oxidizing bacteria on the scalp, in spite of these bacteria being ubiquitous in the environment, it would seem that nitric oxide “poisoning” from this bacterial source is not likely, and perhaps, not even possible. Poisoning due to ingestion of nitrate and subsequent reduction to nitric oxide by bacteria in the gut is known and even occurs in cattle when ingesting feed high in nitrates.

Extreme levels of NO at night may cause hypotension. However, at night, the metabolism is reduced, and there is plenty of excess metabolic capacity to compensate with reflex tachycardia. Limiting extremely high levels of NO to a small slip stream of de-oxygenated blood (that blood which perfuses the scalp), may allow a very high instantaneous concentration to be reached and of which the excess can be destroyed in the lung before systemic hypotension occurs.

Nitric oxide reversibly binds to hemoglobin as nitrosyl hemoglobin (on the heme group) or as S-nitrosyl hemoglobin (on the thiol on cysteine-93 of the beta-globin chain). Nitric oxide can also irreversibly react with oxygenated blood, where the ferrous iron in hemoglobin is oxidized to ferric iron, producing methemoglobin. Methemoglobin is not toxic, however it does not carry oxygen and so if excessive levels build up, there may be insufficient hemoglobin to transport needed oxygen. This occurs at levels of greater than 10 to 20 percent. Individuals with these levels do appear cyanotic. Normal red blood cells have enzymes (NADH-methemoglobin reductase) that rapidly and continuously reduce methemoglobin back to normal hemoglobin. Individuals with defects in their NADH-methemoglobin reductase system are at significant risk of excessive methemoglobin from many sources.

The consumption of nitrates in drinking water can also lead to methemoglobin production. Nitrates in food or drinking water are reduced by intestinal bacteria releasing nitrites and nitric oxide. It is this nitrite and nitric oxide that can oxidize hemoglobin to methemoglobin. The major source of nitrate in the diet is leafy green vegetables, with vegetarians consuming on the order of 0.8 mg nitrate nitrogen/kg/day from vegetables. The most sensitive people to nitrate are infants, and the “no-observed-adverse effect” in drinking water is 1.6 mg nitrate N/kg/day. For a 50 kg vegetarian, this amounts to 2.86 mM NO3 from food and 5.71 mM from water for a total of 8.57 mM NO3. Total ammonia excreted on the scalp amounts to about 3.3 mM.

Nitric oxide is used to treat acute respiratory distress syndrome, and is administered by addition to inhalation air. Provided that other nitrogen oxides are omitted, the only residual concern is the eventual accumulation of excessive quantities of methemoglobin.

NO inhalation does cause a rise in metHb. Clearance of metHb is rapid with a first order time constant of 39-91 minutes. From measured NO absorption rates and metHb clearance rates, it is estimated that continuous breathing of 512 ppm NO would result in a steady state metHb level of 5.7 to 8.2%. Even were large fractions of the urea in sweat to be converted to nitric oxide and absorbed, levels of metHb would be tolerable. It is unlikely that levels of 500 ppm NO would be reached in the scalp biofilm, and unlikely that the diffusion of nitric oxide through the scalp would be more efficient than absorption in the lung. Breathing 500 ppm NO at rest is an exposure of 230 mM/day, or 100 times the total ammonia excreted on the scalp. During NO breathing, essentially all absorbed NO produces metHb.

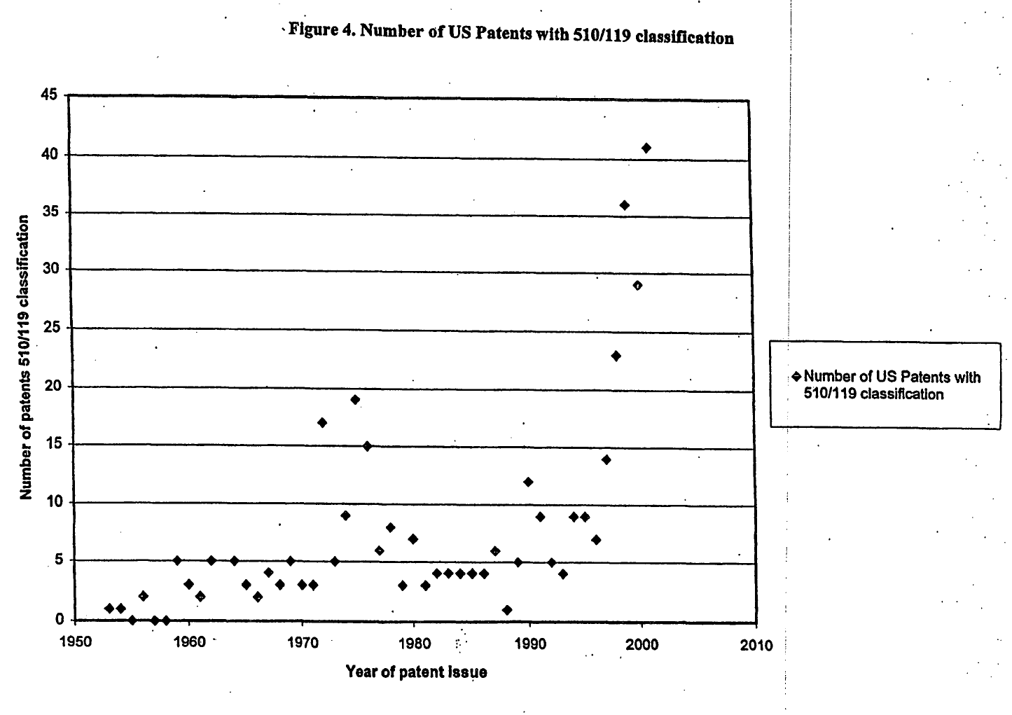

In addition to the changing bathing practices; the current epidemic of obesity may also be due to a change in shampoo technology that occurred in the early 1970's rather than to the introduction of soap. FIG. 4 shows the number of US patents issued on shampoo. Prior to the advent of “conditioning” shampoos, shampooing one's hair was an infrequent exercise because the hair would become unmanageable and unaesthetic.

Nitric oxide can be generated on the skin by the reduction of nitrate in sweat by heterotrophic bacteria. This production of nitric oxide is expected to be substantially lower than that achievable by autotrophic bacteria because the ammonia concentration of sweat is 3 orders of magnitude greater than the nitrate concentration.

In the absence of modern bathing practices, autotrophic ammonia oxidizing bacteria may colonize the skin and scalp and maintain a stable population. Such a stable population has been demonstrated by the inventor, for 8 months on his scalp and now 3 months on the rest of his body. The inventor has refrained from bathing for 3 months, and because the autotrophic bacteria generate significant nitrite and nitric oxide the growth of heterotrophic odor causing bacteria has been suppressed. In spite of not bathing, the inventor remains substantially “odor free”, at least according to reports of co-workers. Daily bathing is only necessary to suppress the odor causing heterotrophic bacteria which can increase many million fold in 24 hours where autotrophic bacteria would only increase a few times.

The widespread use of soap and cleansers is well ingrained in modern society. Preventing the transmission of food born and hand born diseases are important components of the health of modern societies. However, it is only pathogens that are important to avoid. Many heterotrophic bacteria can be opportunistic pathogens, but removal of all bacteria serves no useful purpose. A lack of exposure to non-pathogens may give the immune system nothing to “practice” on and has been suggested as part of the reason for increased asthma and allergies. The notion of “cleanliness is next to Godliness” may have been a useful heuristic when water came from a stream which may have been someone's sewer upstream, when there was no refrigeration of food, and when the consequence of any infection was grave illness or death. Like many good things, bathing and cleanliness may only be good in moderation.

Authentic NO has been shown to cause vasodilatation in vivo for several minutes. The time constants for the decay in the increased blood flow from maximally effective authentic NO, acetylcholine, bradykinin, and SNP were all similar and were all about 1 minute. It is likely that the kinetics of NO removal are concentration dependant, and so the lifetime at concentrations lower than the maximally effective dosages (used in (22)) would be longer. Assuming a concentration independent lifetime of 1 minute, NO from the scalp could contribute and additional concentration of 50 pMolar to 50 nMolar in the blood averaged over a 24 hour period. Basal levels of S-nitrosylated hemoglobin (SNO-Hb) and nitrosyl (heme) hemoglobin (NO-Hb) have been measured at 161±42 nM/l and 150±80 nM/l respectively.

For many physiological processes, it is not the average NO concentration that is of interest, but the instantaneous concentration. If the NO from the scalp were released in ¼ of the time, concentrations may be 4 times higher, or 200 pM to 200 nM. The concentration may also be higher closer to the source of the nitric oxide. Assuming that 1% of the heart output flows through the scalp though probably substantially less, then the NO concentration in that scalp blood would be 100 times higher. That concentration may be high enough to provide a significant driving force for diffusion from the venous blood in the cavernous sinus to the arterial blood flowing through the brain. The levels in the brain could then be several times higher than the average because the transit time from the scalp to the brain is short.

In one embodiment of the invention obesity and diabetes may be greatly reduced by application of autotrophic ammonia oxidizing bacteria to the scalp. I have found from my own experience, that appetite is suppressed, so that dieting and weight control is easier. The ideal treatment method would be to apply autotrophic ammonia oxidizing bacteria to the body and scalp and then not bathe. Those who wish to bathe, may reapply a culture of these bacteria to those bathed parts of the body after bathing. When bathing is done frequently, which necessitates reapplication of the bacteria, it is often desirable to include the normal metabolic products of the bacteria, in particular nitrite and nitrate. These autotrophic ammonia oxidizing bacteria may also be stimulated by applying to the skin nutrients needed by these bacteria such as those nutrients found in American Type Culture Collection standard culture media, including ATCC 1953, ATCC 928, ATCC 1573, ATCC 221, ATCC 929, including, for example, urea, ammonium salts, sodium, potassium, magnesium, calcium, phosphate, chloride, sulfate, trace mineral salts including iron, copper, zinc, cobalt, manganese, molybdenum and buffers. Application of a preparation or solution comprising some or all of those nutrients to the skin and scalp would stimulate the naturally occurring autotrophic bacteria, forming nitrite and nitric oxide without stimulating heterotrophic bacteria.

Nitrobacter are inhibited by elevated pH and by free ammonia. In soil this can lead to the accumulation of nitrite in soil which is quite toxic when compared to nitrate. On the skin, addition of an alkaline agent would raise the pH and inhibit the oxidation of nitrite allowing higher concentrations to develop. Thus using an alkaline compound could serve to increase the concentration of nitrite. Talc while being essentially neutral may contain calcium and magnesium carbonates as impurities. Small amounts of these may then make the skin alkaline when dry, but upon sweating the pH would drop and the increased nitrite would be available for conversion to NO. Inhibiting bacteria such as Nitrobacter that reduce the nitrite concentration on the skin is a useful method to further enhance nitric oxide release. Alternatively, Nitrobacter may be included, which will then increase the production of nitrate. Then other bacteria utilizing this nitrate and the other organic compounds on human skin to form nitrite can be used

Bacteria that are useful in this regard are bacteria that metabolize the normal constituents of human perspiration into NO precursors. These include, for example, urea to nitrite, urea to nitrate, nitrate to nitrite, urea to ammonia, nitrite to nitrate, and ammonia to nitrite. In some cases a mixed culture is preferred. The bacteria may conveniently be applied during or after bathing and may be incorporated into various soaps, topical powders, creams, aerosols, gels and salves. One aspect of the invention contemplates application to body parts that perspire the most, such as, for example, hands, feet, genital area, underarm area, neck and scalp. The major difference between these different areas of the skin is the activity of water. The skin of the hands is much drier than that of the feet, normally covered with socks and shoes, due to the increased exposure of the hands to the drying effects of ambient air. It is contemplated that different strains of bacteria may work best on different areas of the body, and a mixed culture of all the types would allow those that grow best to proliferate and acclimate and become the dominant culture present in a specific area. Clothing may also be worn to change the local microclimate to facilitate the growth of the desired bacteria. For example, wearing a hat may simulate dense hair and help to maintain the scalp in a warmer and moister environment.

Any ammonia oxidizing bacteria may be used in the present invention. In a preferred embodiment, the ammonia oxidizing bacteria may have the following characteristics as are readily known in the art: ability to rapidly metabolize ammonia and urea to nitrite and other NO precursors; non pathogenic; non allergenic; non producer of odoriferous compounds; non producer of malodorous compounds; ability to survive and grow in human sweat; ability to survive and grow under conditions of high salt concentration; and ability to survive and grow under conditions of low water activity.

In one embodiment, bacteria adapted to low water tension environments are placed in close proximity to a subject. Bacteria adapted to low water tension environments are advantageous because a normal skin environment is relatively dry. One example of a moderately halophilic ammonia oxidizing bacteria is Nitrosococcus mobillis described by Hans-Peter Koops, et al. (Arch. Microbiol. 107, 277-282(1976)), incorporated herein by reference. This bacteria has a broad range of growth. For example, while the optimum pH for growth is 7.5, at pH 6.5 it still grows at 33% of its maximal rate. Another more halophilic species, Nitrosococcus halophillus described by H. P. Koops, et al. in arch. Micorbiol. (1990) 154:244-248, incorporated herein by reference, was isolated from saturated salt solutions in a natural salt lake. Nitrosococcus oceanus (ATCC 1907) is halophilic but has an optimum salt concentration intermediate between the other two. The optimum NaCl concentrations for the three are 200, 700, and 500 mM NaCl respectively. N. oceanus however utilizes urea and tolerates ammonia concentrations as high as 1100 mM as ammonium chloride. While growth at optimum conditions is the fastest, similar results may be achieved by using more bacteria. Thus while the optimum pH for growth of N. Mobillis is 7.5, one can achieve the same nitrite production by using 3 times as many bacteria at pH 6.5. Because the quantities of bacteria in the present invention may be large, a number of orders of magnitude larger than that which occurs within 24 hours of bathing, the fact that the pH of the skin is not optimum for these bacteria is not an inhibition to their use. Because N. halophillus was isolated from a saturated salt solution, it should easily survive the relatively moister human skin environment.

In another embodiment; a bacteria that produces nitric oxide directly may be positioned within close proximity to a subject. One example is described in “Production of nitric oxide in Nitrosomonas europaea by reduction of nitrite”, by Armin Remde, et al. in Arch. Microbiol. (1990) 154:187-191, incorporated herein by reference. N. europaea as well as Nitrosovibrio were demonstrated to produce nitric oxide directly. Nitrosovibrio is often found growing on rock where the acid generated causes corrosion. It has been suggested by Poth and Focht, “Dinitrogen production from nitrite by a Nitrosomonas isolate.” (Appl Environ Microbiol 52:957-959), that this reduction of nitrite to volatile nitric oxide is used as a method for the organism to eliminate the toxic nitrite from the environment where the organism is growing, such as the surface of a rock.

Natural bacteria may be used as well as bacteria whose characteristics have been altered through genetic engineering techniques. Bacteria culturing techniques may be used to isolate strains with the above characteristics. A mixture of pure strains may avoid the problems associated with simply culturing bacteria from the skin, which includes the potential growth of pathogens and other bacteria having undesirable characteristics. However, culturing bacteria from the skin and growing them on growth media that simulates the composition of human perspiration may also be effective at increasing the nitric oxide production rate. One method for culturing and isolating such bacteria is to grow them on media containing urea and ammonia plus mineral salts, but without the organic compounds that heterotrophic bacteria utilize, such as sugars and proteins. When isolating autotrophic ammonia and ammonia oxidizing bacteria, it may also be desirable to attempt growth on a heterotrophic media to verify that the autotrophic strain is not contaminated with heterotrophic bacteria.

U.S. Pat. No. 4,720,344 issued to Ganczarczyk, et al. Jan. 19, 1988, and incorporated herein by reference, discloses the conditions that are conducive to the growth of Nitrosomonas but not to Nitrobacter and maximize the conversion of ammonia to nitrite while minimizing the conversion of nitrite to nitrate. This is accomplished most preferably by adjusting the pH and ammonia content of the waste water to levels that are conducive to the growth of Nitrosomonas but not to Nitrobacter and then adjusting the hydraulic retention times in the contacting chambers to less than the recovery time of the inhibited bacteria.

U.S. Pat. No. 5,314,542, incorporated herein by reference, issued to Cassidy, et al. May 24, 1994, and discloses the growth and treatment of bacterial cultures of Nitrosomonas to allow for extended shelf life in a dormant state and subsequent treatment to produce rapid recovery of metabolic activity.

In another embodiment, ammonia oxidizing bacteria cultures to treat heart disease, Alzheimer's Disease, obesity, and diabetes type 2 may also be used where ammonia oxidizing bacteria are grown in a media, concentrated and separated from the media, suspended in sterile water with the proper salt concentration, stored under aseptic conditions, reviving the bacteria through addition of ammonia, and then applied to the skin.

Methods of isolation of bacteria suitable for colonization of human skin are analogous to methods used for the isolation of bacteria suitable for colonization of livestock digestive systems. Scrapings are collected from healthy individuals, inoculated into suitable media, grown and characterized. Steady state continuous culture methods can be used to ensure stability of the culture over time.

Another method of treatment of ammonia oxidizing bacteria cultures, is growing ammonia oxidizing bacteria in a media, concentrating and separating from that media, suspending in sterile water with the proper salt concentration, storing under aseptic conditions, reviving through addition of ammonia, and holding for a period of time for the bacteria to become active.

Ammonia oxidizing bacteria are aerobes which require oxygen for their metabolism and cannot grow in anaerobic conditions. However many of them may also use nitrate as well as oxygen as the terminal electron sink of their metabolic processes. Storage for prolonged periods of time in a sealed container runs the risk of the container becoming anoxic. Nitrate may be added to the fluid in the container so that nitrate can be utilized instead of oxygen for bacteria respiration during storage allowing for non-fluid formulations such as gels and sticks. Bacteria on the interior of such formulations can derive their oxidizing substrate from dissolved nitrate in the absence of dissolved oxygen.

In another embodiment of the present invention, urea, nitrite and nitrate, iron, lactic acid, and salt may be included in a compound comprising the bacteria or applied separately to supplement the skin, because bathing removes these water soluble compounds. The bacteria may also be applied during or after bathing and may be incorporated into various topical powders, creams, sticks, aerosols, and salves. Other compounds may be added to these cosmetic preparations as selected by one skilled in the art of cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid

The ammonia oxidizing bacteria, may be applied to any surface of a subject, such as, for example, skin and hair. In a preferred embodiment, the bacteria is applied to the skin of a subject. In a more preferred embodiment, the ammonia oxidizing bacteria may be applied to the scalp because the scalp provides excellent blood supply. Bacteria may be incorporated into various hair treatments and devices, including conditioners, gels, hair sprays, hair nets, combs, brushes, hats, hair pieces.

Another embodiment of the invention includes analogous methods used for curing meat, since the goal of meat curing is the production of nitric oxide. General properties of nitric oxide, physiological properties, chemical properties, and its role and mechanism of action in food preservation is well described in a book “Nitric Oxide Principals and Actions”, edited by Jack Landcaster, Jr., Academic Press, 1996, incorporated herein by reference. These pickling brines and curing compositions may present little health risk since they are considered safe for human consumption. Heterotrophic bacteria, to achieve a low pH and to produce nitrite from nitrate, are commonly used in meat preserving where nitrate in a pickling brine is reduced to nitrite, releasing NO which reacts with meat to produce the characteristic color and flavor of cured meat. In particular, when nitrite is treated with ascorbic acid, nitric oxide is produced. Nitrite is reduced by ascorbate to generate nitric oxide. Usually in modern meat curing, ascorbate or erythorbate are used with nitrite to generate nitric oxide. In meat preserving, these bacteria are added as a pure culture where they may retard the undesirable growth of disease and spoilage bacteria. Micrococcus varians is sometimes used for this purpose as described in U.S. Pat. No. 4,147,807 issued to Gryczka, et al. At low pH, such as less than 5, nitric oxide is rapidly lost from pickling brines before these chemical reactions can occur. Therefore, a higher pH is recommended for meat preserving. Mixtures of nitrite with erythorbate may be one such example. Applying such bacteria to the skin may enhance the production of nitric oxide on the skin through the reduction of nitrate to nitrite. Some heterotrophic bacteria can produce nitrite from ammonia, but their oxidation of ammonia is substantially slower than that of the autotrophic lithotrophic ammonia oxidizers. Combining meat curing formulations with cosmetic type formulations may achieve a similar benefit. Combining bacteria, urea, and erythorbic acid may be a preferred combination. Other physiologically acceptable acids may be used as well.

An advantage of an embodiment of the invention is that the induced NO production is under physiological control through sweating. Organic nitrates, such as nitroglycerine, are sometimes prescribed for use prior to time of emotional or physical stress. These are the same conditions under which nervous sweating occurs. One aspect of the present invention may be a reduced incidence of heart disease, vascular diseases, impotence, and infertility. Any condition that may be treated through a NO enhancing method may be amenable to treatment with present invention, even where known treatments include administering nitric oxide or NO donor substances orally, topically, sublingually, nasally, by injection, by inhalation. For example, impotence is treated with Viagra, which extends the duration of action of NO. Use of the present invention may reduce the need for the use of agents such as Viagra.

Vaginal use of these bacteria by a woman may also enhance the sexual performance of a male partner by providing additional nitric oxide to her partner's sexual organ during sexual intercourse. Just as the nitrite in saliva provides the basis for the folk remedy for impotence, that of applying saliva to the male sexual organ, the stimulatory effect of the application of saliva to the female genitalia may also have its basis in the nitrite content of saliva. The present invention by enhancing the production of nitric oxide may provide a similar benefit in enhancing the sexual function of both men and women.

### EXAMPLE II

Composition of Nitroceutic Gel:

For approximately 1 liter of gel:

10 g Carbopol ETD 2020
700 g autoclaved Milli-Q+water
mix until uniform
   Neutralize with mixture of 5 moles NaOH+1 mole KOH in one liter water (about 6 ml) pH=7.45. The mixture becomes very viscous as the polymer is neutralized and the hydrophilic chains expand. The gel also has significant shear strength, but yields when this shear strength is exceeded. It is extremely slippery and lubricious.
   300 g grown out culture media, pH=6.0 high nitrite content (tens of mM) high content of living bacteria
pH of mixture=7.1
30 g fresh media (pH=8.4)
pH of final mix=7.1

The addition of the grown out media and the growth media reduces the shear strength of the gel and reduces somewhat the viscosity. This reduction is due to the ionic strength of the added ingredients.

Carbopol EDT 2020 is an “easy to disperse” cross-linked polyacrylic acid copolymer of high molecular weight sold by Noveon, Inc. It is not degraded by bacteria, is considered essentially physiologically “inert”, and has been widely used for 25 years as an aqueous gelling agent for topical cosmetic and lubricant preparations.

Growth Media: The solution was designed to simulate the inorganic composition of human sweat. The salt solution consisted of 1.5 g NaCl, 0. 0.55 g KCl, 0.25 g CaCl2, 0.24 g MgSO4.7H2O, 0.02 g K2HPO4, 2.0 g NaHCO3, 1.5 g NH4Cl one liter milli-Q+water, trace minerals (6 g FeSO4.7H20, 3.5 g CuSO4.5H2O, 0.25 g MnSO4.4H2O, 0.03 g Co(C2H3O2)2.4H2O, 5 g ZnSO4.7H2O, 0.125 g Na2MoO4.2H2O, 0.015 g KI, 2 g EDTA, in 1 liter) were added (1 ml/l of solution) to achieve levels reported in sweat. Co and Mo levels in sweat were unknown but were added to achieve concentrations similar to those in published growth media for ammonia oxidizing bacteria. All other components were within reported ranges for human sweat (which incidentally are fairly close to values for published media). Interestingly, ¼ of ingested iron is reported to be excreted in the sweat. The water content was selected to give both solutions equal osmotic strength. Solutions were prepared by adding the dry salts to autoclaved mQ+water immediately prior to use. The pH range for sweat is 4 to 6.8. In this pH range most ammonia is present as ammonium ion which is unavailable to the ammonia oxidizing bacteria. Urea should be available even at low pH. Earlier studies demonstrated that the urea is rapidly (hours) hydrolyzed to ammonia, so ammonium chloride and pH 7.8 was used to reduce the need for pH adjustment and to provide for ammonia availability. Bicarbonate was chosen as the major non-chloride anion instead of lactate to reduce the possible growth of heterotrophic bacteria. The initial pH of the nutrient solution was about 7.8, however, fairly rapidly, a precipitate formed and the pH would rise (due to formation of CaCO3). Bacteria accelerated this process, presumably by supplying enzymes that catalyze the conversion of bicarbonate into carbonate.

The grown out media was the growth media which had been inoculated with autotrophic ammonia oxidizing bacteria and allowed to grow. The mixture had high levels of nitrite (tens of mM) and had a pH of about 6. The pH was reduced solely by bacterial action.

It is believed that only autotrophic ammonia oxidizing bacteria are living in the gel, although the gel was not produced under sterile conditions and was not been tested for the presence of any bacteria. The culture media for the bacteria was organic free, so the only source of organic carbon for heterotrophic organisms is the autotrophic bacteria themselves. The only source of nitrite in the culture is the bacteria themselves, and they have produced quite high levels of nitrite, indicating their presence. The levels of nitrite in the mature growth media are quite high, and may inhibit many different types of bacteria. The gel has been used topically by a number of subjects with no adverse reactions reported. It has been used on minor wounds where it apparently inhibited infection and promoted healing. The maximum nitrite level that may be present is about 10 mM, thus 10 ml of the gel could release at most 50 μM of NO (half a mole of NO per mole of nitrite). The gel is expected to release prompt NO through decomposition of nitrite, and release sustained NO through the metabolic activity of the autotrophic ammonia oxidizing bacteria that are present. For topical application, it is anticipated that the urea in sweat and other bodily secretions would serve as substrate for these bacteria and that release of sweat is a physiological mechanism whereby the body can regulate the metabolic activity of these bacteria living on the skin.

The gel was analyzed for nitric oxide using a potentiometric NO sensor. The measurement was made about 2 months after the gel was produced and stored at ambient conditions. The NO content was approximately 2.2 μM. This measurement demonstrates that the gel remains active for a significant shelf life.

Topical use of this gel has demonstrated a number of positive health effects. It is a sexual aid. It has been demonstrated to potentiate sexual arousal for both men and women when applied topically to the genitalia prior to sexual intercourse. It is believed that this is mediated through the action of nitric oxide. The well known sexual aid, Viagra, aids in the achievement of male erection through the action of Viagra on the enzyme phosphodiesterase type 5. In the relevant anatomy of the male sexual organ, nitric oxide stimulates guanylyl cyclase and so stimulates the production of cyclic GMP. cGMP causes the relaxation of various smooth muscles which cause parts of the male sexual organ to become engorged. cGMP is degraded by phosphodiesterase type 5, and Viagra, by inhibiting this enzyme, prolongs the action of NO. Phosphodiesterase type 5 has been found in structures in the genitalia of both men and women. Nitric oxide is well known to have stimulatory effects on the male sexual organ, and similar effects on the female sexual organs are not unexpected. Neuronal nitric oxide synthase (nNOS) immunoreactivity has been found in nerves within the glans clitoris and corpora cavernosa of the clitoris, also endothelial nitric oxide synthase (eNOS), an enzyme that can be stimulated by shear stress, was found in vascular tissue located in the human clitoris suggesting that nitric oxide is necessary for proper functioning of this organ. Type 5 phosphodiesterase (the enzyme inhibited by Viagra) is also expressed in the human vagina. Studies have shown that the erectile tissue in the clitoris is stimulated and engorged by nitric oxide and numerous products claiming stimulatory nitric oxide mediated effects are available on the internet. The common folk remedy for impotence, applying saliva to the penis, utilizes the stimulatory effects of salivary derived nitric oxide.

Topical use of the gel on other parts of the body has also demonstrated health effects. It has had good activity against the fungal infection athletes' foot. It has had good action against the bacterial infection of acne, and good activity against the viral infection which causes plantar warts. Nitric oxide has been widely shown to have very good anti-microbial action against many disease causing organisms. Indeed, the body's immune system utilizes nitric oxide generated by iNOS as an anti-microbial agent.

I have also found that applying these bacteria to the skin suppresses body odor. I have applied a culture of these bacteria to my skin in May, and have not bathed for over 6 months. During the summer months, body odor was completely suppressed. During winter, odor did start to increase, however, by inducing sweating through the wearing of additional layers of clothing (a sweater), the autotrophic bacteria were nourished and the heterotrophic bacteria were suppressed. The suppression is quite prompt and quite dramatic. Another aspect of the invention includes the use of the bacteria to inhibit the growth of heterotrophic bacteria. Body odor derives, in part, from bacterial metabolites on the skin. The development of skin odor in a day or so indicates that fast growing heterotrophic bacteria generate the odoriferous compounds. Ammonia oxidizing bacteria, by inhibiting the growth of the heterotrophic bacteria, may decrease the odor produced. Thus the present invention may also be used to reduce body odor, and may be used alone or in conjunction with other deodorant type cosmetic preparations.

Suppression of heterotrophic bacteria on the skin is useful for the prevention of infection when the skin is damaged through trauma or burns. I have applied the gel to small scrapes and cuts, which have healed quickly and with no infection or other side effects. The prevention of infection for patients with burns over large areas of the body is a very important aspect of the treatment of burn victims. There has been no reported case of infection by these autotrophic bacteria, and it is likely that they are incapable of causing infection, even in immunocompromised individuals. These bacteria are obligate autotrophs, they are incapable of utilizing organic substrates for either growth or as an energy source.

Wound healing follows a certain progressionⁱ, many of the steps of which are mediated through nitric oxide. The first event is the formation of a fibrous clot to prevent blood loss. Then immune cells infiltrate the clot and begin to generate large quantities of nitric oxide so as to sterilize the wound. When the nitric oxide reaches a certain level, then the actual “healing” may begin. Granular tissue forms leading to the laying down of matrix and eventually scar tissue. Vascular regrowth is also controlled in large part through nitric oxide. Supplying nitric oxide with autotrophic bacteria can allow the wounded skin to utilize what metabolic capacity it has for other purposes.

Application of these bacteria to the scalp has also lead to the growth of hair on areas made bare through male pattern baldness. There is some association of baldness with heart disease, which has been mainly attributed to male sex hormones.

I have also found that my appetite is reduced and in the approximately 1 year since applying these bacteria to my scalp and body, I have lost about 25 pounds, or about 12% of my original body weight. I have not exercised extensively, but have simply reduced the amount I eaten. Because of the reduction in appetite, it has not at all been difficult to achieve this weight loss.

Morning sickness is a common occurrence during the early stages of pregnancy. It is characterized by reduced appetite and a reduced interest in eating. Interestingly, epidemiologic studies find that pregnancies characterized by morning sickness tend to have “better” outcomes than non-symptomatic pregnancies. Nitric oxide production is increased in normal pregnancy and decreases in preeclampsia. A polymorphism in eNOS is associated with preeclampsia. Abnormal nitric oxide synthesis has been demonstrated in some preeclampsia pregnancies. I suggest that the appetite reduction that accompanies morning sickness is due to elevated nitric oxide levels, and that these elevated nitric oxide levels in early pregnancy facilitate the vascular remodeling of the uterus which is necessary for good vascular exchange between the growing placenta and the uterus.

Application of the bacteria of the present invention may also be useful in the prevention of preeclampsia. Pre-eclampsia in Norway, characterized in part by maternal blood pressure exceeding 140/90 mmHg, peaks in December and has a minimum in Aug. Transdermal administration of nitric oxide donors to women with preeclampsia improves uteroplacental circulation. Nitric oxide metabolism is reported to be dysfunctional in women with preeclampsia. I suggest that the seasonality of preeclampsia and the reduced incidence in summer has to do with increased sweating during the summer, and increased nitric oxide from sweat derived nitrate being reduced to nitric oxide, or to nitric oxide production from autotrophic ammonia oxidizing bacteria on the scalp. In that humans evolved in Africa, where it is warm year round, and where sweat residues may also accumulate year round to nourish autotrophic ammonia oxidizing bacteria on the scalp, restoring the natural populations of these bacteria.

Preeclampsia may be prevented by applying autotrophic bacteria to the skin of a pregnant woman, especially a woman at risk for developing preeclampsia. It should be recognized that treatment with these bacteria, which will increase the basal level of nitric oxide, will perhaps make morning sickness worse. It is important to treat the pregnant woman early in her pregnancy so that the vascular remodeling that requires nitric oxide can occur before the growing fetus has high metabolic demands.

All bodily secretions contain significant urea (adult plasma 5 mM, sweat 10 mM, urine 200 mM, saliva 4 mM, seminal plasma 12 mM, breast milk 3 MM)ⁱⁱ, and so in principle all bodily secretions can provide these bacteria with substrate with which to make nitric oxide.

Reductions in appetite are observed in a number of circumstances. Exposure to high altitude causes a profound reduction in appetite for both humans and experimental animals. Many infectious diseases and cancers are often characterized by cachexia, a profound and eventually debilitating weight loss due to an inability to eat. I suggest that the suppression of appetite in these diverse examples has a common cause, that of an increased basal nitric oxide level compared to the ambient oxygen level. The major oxygen sensing enzymes are heme containing enzymes where molecular oxygen in bound to an iron atom coordinated in a porphyrin ring. In addition to binding oxygen, hemes also bind nitric oxide and carbon monoxide. Nitric oxide is well known to compete with oxygen on cytochrome oxidase where it inhibits cytochrome oxidase especially at low oxygen levels.

All bodily secretions contain significant urea (adult plasma 5 mM, sweat 10 mM, urine 200 mM, saliva 4 mM, seminal plasma 12 mM, breast milk 3 mM)ⁱⁱⁱ, and so in principle all bodily secretions can provide these bacteria with substrate with which to make nitric oxide.

Sickle cell disease is a disorder where hemoglobin has a structural defect which allows deoxygenated hemoglobin to polymerize and convert the red blood cells from their normal compliant texture to rigid (and sickle shape). In that this occurs under conditions of low oxygen tension, it primarily occurs in the capillary beds where oxygen is removed from the blood, and where the vessel diameter is the smallest. Because the sickled cells are rigid, they do not deform and flow readily through the small capillaries. As these are blocked, oxygen transport to the blocked vessel is diminished, leading to a further lowering of oxygen levels and to an exacerbation of the blockage, a positive feedback.

Hydroxyurea is an approved treatment for sickle cell disease and it has been demonstrated in vitro that the addition of hydroxyurea to oxyhemoglobin, deoxyhemoglobin or methemoglobin, causes the production of nitrosylhemoglobin.

Increased basal nitric oxide has several positive effects on sickle cell disease. Nitric oxide dilates blood vessels and so reduces the tendency for them to clog up with sickled cells, it reduces the tendency for blood cells to aggregate together, and it modifies hemoglobin by forming nitrosylhemoglobin which has a reduced tendency to polymerize. Even a slight delay in the onset of polymerization can have an important influence on outcomes because of the positive feedback that occurs once sickling starts.

The present invention may also be useful for the treatment of sickle cell disease. Acute sickle cell disease shows seasonality with the peak season during cold weather and reduced incidence in hot weather. I suggest that this observed seasonality is due to increased sweat residues present during hot weather, and the resulting increased nitric oxide from either autotrophic of heterotrophic bacteria. Using autotrophic bacteria in the manner of the present invention will provide greater benefit all year long.

Subjects need not have clinical symptoms of any of these disorders in order to benefit from the present invention. The invention may be used as a preventative measure along the same lines as proper diet, taking vitamins, exercising, or as bathing in general. Because disorders are related through the common action of the vasodilator NO, one can use the invention for heart disease prevention and receive a therapeutic value for impotence. Because impotence is a disorder that is often stigmatized, an impotence treatment that can be disguised as a general health tonic is advantageous.

In another embodiment of the invention, the bacteria may be applied to the surface of non-human vertebrates. Domesticated animals such as horses, dogs, pigs, and chickens are seen to roll in and cover themselves with dirt. In that urea is an abundant compound in urine and manure, bacteria adapted to living in barnyard soil may be expected to be rapid metabolizers of urea into nitrite. Wild animals also cover themselves with dirt. A component of such behavior may likely be the inoculation of the skin or fur with bacteria that will metabolize sweat components into NO and NO precursors. Using a substantially pure culture of such bacteria may improve the health of domesticated animals and facilitate their growth. Ammonia is often present in large amounts in animal feed lot areas. Bacteria that would metabolize ammonia into NO or NO precursors would reduce the ill effects of ambient ammonia and improve the economics of intensive animal farming. Other subjects include, but are not limited to, vertebrates such as, domesticated, laboratory, transgenic, chimeric, and zoo animals such as, horse, pig, cow, dog, cat, goat, sheep, buffalo, donkey, mule, elephant, cat, wolf, camel, llama, chicken, turkey, primates, ungulates, rodents, chimpanzees, gorilla, orangutan, mice, rats, and rabbits.

The practice of some animals, to deposit their urine and feces in a single location can be seen as their instinctive production of a rich environment for culturing and proliferation of nitrite producing bacteria. That animals instinctively exhibit behaviors that re-inoculate their skins with these bacteria may indicate that these bacteria may be readily lost from the skin and that re-inoculation may be necessary for animals. In that humans typically bathe more frequently than animals, the human need for re-inoculation may be greater.

Foundering or aquine laminitis is treated through application of a nitric oxide donor to the feet and hoof region. Nitroglycerine has been used, as have other nitric oxide donors. Horses instinctively accomplish this in the wild by urinating in the mud, allowing nitrite forming bacteria to proliferate, and walking through this mud containing the nitrite producing active cultures. Modern stable practices call for good house keeping and the elimination of any accumulation of urine and feces where horses walk. All hoofed animals are subject to similar disorders of the feet and hooves. Thus all hoofed animals may benefit from application of the suitable ammonia oxidizing bacteria.

Additionally, application of an appropriate ammonia oxidizing bacteria to a horse's skin may have the effect of increasing the natural production of nitric oxide during exercise. Nitric oxide may diffuse through the horse's skin and be absorbed into the blood where it would circulate resulting in systemic effects. Some nitric oxide may also be released into the air around the horse and may be inhaled. Presumably decreased pressure drop translates into increased maximal flow of air and blood in the lungs, and hence increased maximal exercise performance. Achieving this increased performance through natural means would be advantageous in horse racing. Similarly racing gray hounds, draft animals, beasts of burden, and animals under stress may also have their nitric oxide production enhanced. Human athletes may similarly enhance their performance by utilizing skin bacteria to augment nitric oxide production before, during and after exercise. Typical athletic events include, for example foot races, weight lifting, bicycle race or practice, football game or practice, soccer game or practice, basket ball game or practice, baseball game or practice, golf game or practice, mountain climbing, boxing match or practice, hockey game or practice, and tennis match or practice.

In another embodiment of the invention, ammonia oxidizing bacteria may be positioned in close proximity to a surface of a subject by being applied directly or indirectly to the surface of the subject. Suitable bacteria may be positioned in close proximity to the surface of the subject by being indirectly applied by application to articles with which the surface of the subject comes into contact, such as, for example, bedding products such as straw, wood shavings, pillows, sheets, habitat enclosures, stalls, brushes, combs, and mattresses. Similarly suitable bacteria can be added to litter box products so that when the animal comes into contact with the litter and litter box, the animal subject will be in close proximity to the bacteria. As an added feature of such litter box products the urea in urine may be oxidized to non-volatile products and the ammonia smell of litter boxes will be reduced. Rather than give off ammonia, the litter boxes would give off nitric oxide which would enhance the pulmonary function of animals and humans in the vicinity, as well as provide systemic effects.

In one aspect of the invention, an article is treated with ammonia oxidizing bacteria. For example the article may be coated or impregnated with the bacteria. In a preferred embodiment, the article treated with the bacteria, contacts a surface of a subject, such as, for example, clothing, collar, and saddle.

Articles contacting the surface of a human subject, such as a diaper, may be treated with ammonia oxidizing bacteria. Because diapers are designed to hold and contain urine and feces produced by incontinent individuals, the urea in urine and feces can be hydrolyzed by skin and fecal bacteria to form free ammonia which is irritating and may cause diaper rash. Incorporation of bacteria that metabolize urea into nitrite or nitrate may avoid the release of free ammonia and may release nitrite and ultimately NO which may aid in the maintenance of healthy skin for both children and incontinent adults. The release of nitric oxide in diapers may also have anti-microbial effects on disease causing organisms present in human feces. This effect may continue even after disposable diapers are disposed of as waste and may reduce the incidence of transmission of disease through contact with soiled disposable diapers. The addition of the ammonia oxidizing bacteria to the diaper is beneficial when realizing that cleaning a soiled infant can remove ammonia oxidizing bacteria faster than they can proliferate, leaving only heterotrophic urea hydrolyzing bacteria on the skin. The epidemic of infant deaths due to Sudden Infant Death Syndrome, or SIDS, in the 1980's was approximately coincident with the widespread use of disposable diapers. The “back to sleep” program where infants are put to sleep on their backs has greatly reduced the incidence of SIDS. The mechanism of the causal relationship between back sleeping and low SIDS incidence remains elusive, however, it may be due to the increased contact of infant skin with urine during sleep occurring while the infant is lying on its back. Victims of SIDS are often found with sweat soaked bed clothes which may be due to due to the infant's vain attempt to increase nitric oxide formation during asphyxiation by sweating, rather than due to overheating as is conventionally thought.

Another article of clothing that can be so treated is the tampon. During a woman's menstrual period, secretions are generated which under certain circumstances can support the growth of heterotrophic disease causing bacteria such as those that cause toxic shock. Just as topically applied acidified nitrite has been shown to be curative for yeast infections of the skin, it is expected that vaginal application of these bacteria should be curative and preventative of vaginal yeast infections. By rendering the vagina less hospitable to disease causing organisms, the incidence of transmission of sexually transmitted diseases can be reduced.

Other articles of clothing such as, for example, shoes, shoe inserts, pajamas, sneakers, belts, hats, shirts, underwear, athletic garments, helmets, towels, gloves, socks, bandages, and the like, may also be treated with ammonia oxidizing bacteria. Bedding, including sheets, pillows, pillow cases, and blankets may also be treated with the bacteria. In one embodiment of the invention, areas of skin that cannot be washed for a period of time may also be contacted with ammonia oxidizing bacteria. Specifically, skin enclosed in orthopedic casts which immobilize injured limbs during the healing process, and areas in proximity to injuries that must be kept dry for proper healing such as stitched wounds may benefit from contact with the ammonia oxidizing bacteria.

It is contemplated that articles worn about the head and scalp may be treated with ammonia oxidizing bacteria. Nitric oxide formed on the hair, away from the skin surface, may be captured in a hat, scarf or face mask and directed into inhaled air.

Individuals having a reduced bathing frequency, such as astronauts, submarine crew members, military personnel during a campaign, civilian workers in remote locations, refugees, bedridden individuals and many others may maintain healthier skin by maintaining skin bacteria according to the present invention. Bed sores are a common factor deriving from disturbances to blood flow. It is expected that the present invention may augment and normalize inadequate circulation problems.

In another embodiment, garments such as, for example, condoms, and codpieces may be treated with the proper bacteria. The stimulation from nitric oxide generation through wearing the articles may be beneficial for male subjects contemplating sexual acts. Similarly, the treatment of fabric coverings of furniture used for sexual activities would also be advantageous, such as, for example, sheets, blankets, slip covers, pillow cases.

Ammonia oxidizing bacteria may be located on a surface of the article directly contacting the surface of the subject. Alternatively, the bacteria may be exposed to bodily fluids but not directly in contact the surface of the subject. In particular, a diaper, tampon, or bandage may have an inner layer treated with the ammonia oxidizing bacteria, and at least one layer that is permeable to bodily fluids, nitric oxide, and or nitric oxide precursors. These layers need not be permeable to bacteria. Because the ammonia oxidizing bacteria cannot utilize compounds other than ammonia for energy, they cannot infect a wound. Although they may be allergenic, the inhibition of growth of heterotrophic bacteria may outweigh the potential for allergy.

It is contemplated that different bacteria will be suitable for different applications.

Thus bacteria adapted for very high levels of nitrite production may be ideal for use in diapers, animal bedding, and other non-contact applications. High nitrite levels would also be useful for protecting skin from infections during extended safaris in tropical environments, for military type applications, or for the enhancement of performance of elite athletes, human and non-human vertebrate.

While it is expected that the autotrophic ammonia oxidizing bacteria will be the most active at producing nitric oxide and nitric oxide precursors, other bacteria producing lessor amounts may be used as well. These may be desired in some circumstances when, for example, better control of the nitric oxide production is needed. Other bacteria can be included for other purposes, such as, for example, to control the pH through production of acid.

Further modification and equivalents herein disclose will occur to persons skilled in the art using no more than routine experimentation, and all such modifications and equivalents are believed to be within the spirit and scope of the invention as defined by the following claims.

All references, patents and patent publications that are recited in this application are incorporated in their entirety herein by reference.

1. A method treating a subject who has developed or is at risk of developing at least one of high blood pressure, Alzheimer's Disease, Obesity, Diabetes Type II, Sickle Cell Anemia, Preeclampia, Sudden Infant Death Syndrome, or Vascular Disease comprising:
positioning ammonia oxidizing bacteria in close proximity to the subject.

2. The method of claim 1, wherein the subject does not otherwise need nitric oxide.

3. The method of claim 1, wherein the act of positioning ammonia oxidizing bacteria in close proximity to the subject comprises applying the ammonia oxidizing bacteria to a surface of the subject in an effective amount to cause the bacteria to metabolize any of ammonia, ammonium salts, or urea on the surface into any of nitric oxide, nitric oxide precursors, or combinations thereof.

4. The method of claim 1 wherein the act of positioning the bacteria occurs prior to sleep.

5. The method of claim 3, wherein the act of positioning the bacteria comprises applying the bacteria in a suitable carrier.

6. The method of claim 1, wherein the act of positioning the bacteria comprises positioning a bacteria selected from the group consisting of any of Nitrosomonas, Nitrosococcus, Nitrosospira, Nitrosocystis, Nitrosolobus, Nitrosovibrio, and combinations thereof.

7. The method of claim 3, wherein the act of applying the bacteria to a surface comprises applying the bacteria to skin, hair, or a combination thereof.

8. The method of claim 1, wherein the act of applying the bacteria comprises applying a substantially pure bacteria.

9. The method of claim 1, wherein the act of applying the bacteria comprises:
applying the bacteria to an article; and
contacting the article with the surface of the subject.

10. The method of claim 1, further comprising the act of applying a compound selected from any of a component of perspiration, urea, nitrite, lactic acid, nitrate, salt, iron salts, ammonium salts, and combinations thereof, to the surface of the subject.

11. The method of claim 6, further comprising:
administering to the surface of the subject at least one of urea or metal salts to the surface of the subject in an effective amount to stimulate the growth of the bacteria.

12. The method of claim 10, wherein the act of contacting the article with the surface of the subject further comprises contacting the bacteria with the surface of the subject.

13. The method of claim 1, wherein the act of administering the bacteria comprises applying the bacteria to a subject that is a non-human vertebrate.

14. A preparation for treating a subject who has developed or is at risk of developing at least one of high blood pressure, Alzheimer's Disease, Obesity, Diabetes Type II, Sickle Cell Anemia, Preeclampia, Sudden Infant Death Syndrome, or Vascular Disease comprising:
an active culture of nitric oxide producing bacteria.

15. The preparation of claim 14, where said nitric oxide producing bacteria are autotrophic ammonia oxidizing bacteria.

16. The preparation of claim 14, where said nitric oxide producing bacteria are combined with a substrate from which said bacteria produce nitric oxide.

17. The preparation of claim 14, wherein the nitric oxide producing bacteria are combined with a substrate chosen from the list of: ammonia, ammonium salts, urea, nitrite salts, nitrate salts.

18. The preparation of claim 14, wherein the preparation is any of a cosmetic composition, a body deodorant, or an athletic preparation.

19. The preparation of claim 15, wherein the bacteria is selected from any of Nitrosomonas, Nitrosococcus, Nitrosospira, Nitrosocystis, Nitrosolobus, Nitrosovibrio, and combinations thereof.

20. The preparation of claim 18, further comprising at least one component selected from any of water, mineral oil, coloring agent, perfume, aloe, glycerin, sodium chloride, pH buffers, UV absorbing agents, silicone oil, natural oil, vitamin E, herbal concentrates, Lactic acid, citric acid, talc, clay, calcium carbonate, magnesium carbonate, zinc oxide, starch, urea, nitrite, nitrate, iron salts, ammonium salts, and combinations thereof.

21. The preparation of claim 18, wherein the preparation is any of powder, cream, stick, aerosol, or salve.

22. The preparation of claim 14, wherein the subject is a human being.

23. The preparation of claim 16, further comprising:
at least one compound selected from any of urea, ammonium salts, sodium, potassium, magnesium, calcium, phosphate, chloride, sulfate, trace mineral salts, iron, copper, zinc, cobalt, manganese, molybdenum, buffers, and combinations thereof.

24. A method of increasing basal nitric oxide in a subject comprising applying positioning ammonia oxidizing bacteria in close proximity to the subject.

25. The method of claim 24, wherein the act of positioning the bacteria comprises applying the bacteria in a suitable carrier.

26. The method of claim 24, wherein the act of positioning the bacteria comprises positioning a bacteria selected from the group consisting of any of Nitrosomonas, Nitrosococcus, Nitrosospira, Nitrosocystis, Nitrosolobus, Nitrosovibrio, and combinations thereof.

27. The method of claim 24, wherein the act of applying the bacteria to a surface comprises applying the bacteria to skin, hair, or a combination thereof.

28. The method of claim 24, further comprising the act of applying a compound selected from any of a component of perspiration, urea, nitrite, lactic acid, nitrate, salt, iron salts, ammonium salts, and combinations thereof, to the surface of the subject.

29. A method of treating a wound in a subject comprising applying the ammonia oxidizing bacteria to a wound of the subject in an effective amount to cause the bacteria to metabolize any of ammonia, ammonium salts, or urea on the surface into any of nitric oxide, nitric oxide precursors, or combinations thereof.

30. The method of claim 29, wherein the bacteria is selected from the group consisting of any of Nitrosomonas, Nitrosococcus, Nitrosospira, Nitrosocystis, Nitrosolobus, Nitrosovibrio, and combinations thereof.
